(19) United States
(12) Patent Application Publication
(10) Pub. No.: US 2024/0402274 A1
(43) Pub. Date: Dec. 5, 2024

(54) METHOD AND APPARATUS FOR PREDICTING PERSISTENT POSTCONCUSSIVE NEUROPSYCHIATRIC SYMPTOMS USING THALAMOCORTICAL COHERENCE

(71) Applicant: TAIPEI MEDICAL UNIVERSITY, TAIPEI CITY (TW)

(72) Inventors: YI-TIEN LI, TAIPEI CITY (TW); CHENG-YU CHEN, TAIPEI CITY (TW)

(21) Appl. No.: 18/326,004

(22) Filed: May 31, 2023

(51) Int. Cl. G01R 33/48; A61B 5/00; G16H 10/20; G16H 50/30

(52) U.S. Cl. CPC G01R 33/4806; A61B 5/4064; A61B 5/7275; G16H 10/20; G16H 50/30

(57) ABSTRACT

The present disclosure provides a method and an apparatus for predicting persistent post-concussive neuropsychiatric symptoms based on thalamocortical coherence. The method includes the following steps: receiving a first set of biomarkers of a plurality of thalamic sub-nuclei of a patient; calculating a first coherence matrix from the first set of biomarkers; and predicting a postconcussive symptom score of the patient for a given time through a machine learning-based predictive model based on the first coherence matrix.

### FIELD OF THE INVENTION

The present disclosure relates to postconcussive neuropsychiatric symptom prediction, and, in particular, to a method and an apparatus for predicting persistent postconcussive neuropsychiatric symptoms using thalamocortical coherence.

### BACKGROUND

Concussion, a mild traumatic brain injury (mTBI) caused by blows to the head moving the brain to impact the skull, typically presents with neurological dysfunctions such as headache, sleep disturbance, depression, and cognitive impairment, and typically resolves within a few months. However, a subset of patients may have persistent postconcussive symptoms (PCS) over years, although the pathomechanism remains poorly understood. There are growing concerns regarding the potential long-term sequelae of concussion including chronic neurologic issues related to stepwise neurocognitive impairment in later life. Hence, clarifying the pathomechanisms underlying various persistent PCS is essential to early identification of biomarkers of prolonged PCS.

### SUMMARY OF THE DISCLOSURE

In an aspect of the present disclosure, a method for predicting persistent post-concussive neuropsychiatric symptoms based on thalamocortical coherence is provided. The method includes the following steps: receiving a first set of biomarkers of a plurality of thalamic sub-nuclei of a patient; calculating a first coherence matrix from the first set of biomarkers; and predicting a postconcussive symptom score of the patient for a given time through a machine learning-based predictive model based on the first coherence matrix.

In another aspect of the present disclosure, an apparatus for predicting persistent post-concussive neuropsychiatric symptoms based on thalamocortical coherence is provided. The apparatus includes: at least one memory having computer executable instructions stored therein; and at least one processor coupled to the at least one memory. The computer executable instructions cause the at least one processor to perform operations including: receiving a first set of biomarkers of a plurality of thalamic sub-nuclei of a patient; calculating a first coherence matrix from the first set of biomarkers; and predicting a postconcussive symptom score of the patient for a given time through a machine learning-based predictive model based on the first coherence matrix.

### BRIEF DESCRIPTION OF THE DRAWINGS

Aspects of the present disclosure are best understood from the following detailed description when read with the accompanying figures. It is noted that in accordance with the standard practice in the industry, various features are not drawn to scale. In fact, the dimensions of the various features may be arbitrarily increased or reduced for clarity of discussion.

FIG. 1 is a perspective view of a thalamus and nuclear groups therein in accordance with an embodiment of the present disclosure.

FIG. 2 is a diagram showing the thalamic reticular nucleus (TRN) with respect to the thalamus and cortex in accordance with an embodiment of the present disclosure.

FIG. 3 is a flowchart of participant recruitment in accordance with an embodiment of the present disclosure.

FIGS. 4A-4B are diagrams illustrating thalamocortical tracts injury and peri-thalamic injury centered around the thalamic reticular nucleus (TRN) on diffusion tensor imaging (DTI) in accordance with the embodiment of FIG. 3

FIG. 5A is a diagram illustrating significantly increased within-thalamic resting-state functional connectivity in patients compared with the HCs in baseline assessment and follow-up in accordance with an embodiment of the present disclosure.

FIGS. 5B-1 to 5B-3 are diagrams illustrating increase in coherence between thalami and almost all cortical regions in the low-frequency band in patients compared with HCs in baseline assessment and follow-up in accordance with the embodiment of FIG. 5A.

FIGS. 5C-1 to 5C-2 are diagrams illustrating fMRI signal in different thalamic subdivisions for a representative HC in accordance with the embodiment of FIG. 5A.

FIGS. 5D-1 to 5D-2 are diagrams illustrating within-thalamic functional connectivity matrix and thalamocortical coherence matrix for a representative HC in accordance with the embodiment of FIG. 5A.

FIGS. 5E-1 to 5E-2 are diagrams illustrating fMRI signal in different thalamic subdivisions for a representative patient with mTBI in accordance with the embodiment of FIG. 5A

FIGS. 5F-1 to 5F-2 are diagrams illustrating within-thalamic functional connectivity matrix and thalamocortical coherence matrix for a representative patient with mild traumatic brain injury (mTBI) in accordance with the embodiment of FIG. 5A.

FIGS. 6A-1, 6B-1, and 6C-1 illustrate the within-thalamic functional connectivity matrices for a representative symptom-resolved patient with mild traumatic brain injury (mTBI) in accordance with the embodiment of FIG. 5A.

FIGS. 6A-2, 6B-2, and 6C-2 illustrate the thalamocortical functional coherence matrix for a representative symptom-resolved patient with mild traumatic brain injury (mTBI) in accordance with the embodiment of FIG. 5A.

FIGS. 6D-1, 6E-1, and 6F-1 illustrates the within-thalamic functional connectivity matrix (left) for a representative symptom-prolonged patient with mild traumatic brain injury (mTBI) in accordance with the embodiment of FIG. 5A.

FIGS. 6D-2, 6E-2, and 6F-2 illustrate the thalamocortical functional coherence matrix for a representative symptom-prolonged patient with mild traumatic brain injury (mTBI) in accordance with the embodiment of FIG. 5A.

FIGS. 6G-1 to 6G-2 are diagrams illustrating the distribution of the PCSQ scores of the symptom-resolved patients and symptom-prolonged patients at 1-year follow-up in accordance with the embodiment of the FIGS. 6A-6C.

FIGS. 6H-1 to 6H-2 are diagrams illustrating the distribution of the PCSQ scores of the symptom-resolved patients and symptom-prolonged patients at 2-year follow-up in accordance with the embodiment of the FIGS. 6D-6F.

FIG. 7 is a flowchart of a method for predicting persistent postconcussive symptoms using thalamocortical coherence in accordance with an embodiment of the present disclosure.

FIG. 8 is a schematic diagram showing a computer device 800 according to some embodiments of the present disclosure.

Corresponding numerals and symbols in the different figures generally refer to corresponding parts unless otherwise indicated. The figures are drawn to clearly illustrate the relevant aspects of the various embodiments and are not necessarily drawn to scale.

### DETAILED DESCRIPTION

The following disclosure provides many different embodiments, or examples, for implementing different features of the provided subject matter. Specific examples of operations, components, and arrangements are described below to simplify the present disclosure. These are, of course, merely examples and are not intended to be limiting. For example, a first operation performed before or after a second operation in the description may include embodiments in which the first and second operations are performed together, and may also include embodiments in which additional operations may be performed between the first and second operations. For example, the formation of a first feature over, on or in a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Time relative terms, such as “prior to,” “before,” “posterior to,” “after” and the like, may be used herein for ease of description to describe one operations or feature's relationship to another operation(s) or feature(s) as illustrated in the figures. The time relative terms are intended to encompass different sequences of the operations depicted in the figures. Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Relative terms for connections, such as “connect,” “connected,” “connection,” “couple,” “coupled,” “in communication,” and the like, may be used herein for ease of description to describe an operational connection, coupling, or linking one between two elements or features. The relative terms for connections are intended to encompass different connections, coupling, or linking of the devices or components. The devices or components may be directly or indirectly connected, coupled, or linked to one another through, for example, another set of components. The devices or components may be wired and/or wireless connected, coupled, or linked with each other.

As used herein, the singular terms “a,” “an,” and “the” may include plural referents unless the context clearly indicates otherwise. For example, reference to a device may include multiple devices unless the context clearly indicates otherwise. The terms “comprising” and “including” may indicate the existences of the described features, integers, steps, operations, elements, and/or components, but may not exclude the existences of combinations of one or more of the features, integers, steps, operations, elements, and/or components. The term “and/or” may include any or all combinations of one or more listed items.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

The nature and use of the embodiments are discussed in detail as follows. It should be appreciated, however, that the present disclosure provides many applicable inventive concepts that can be embodied in a wide variety of specific contexts. The specific embodiments discussed are merely illustrative of specific ways to embody and use the disclosure, without limiting the scope thereof.

On the basis of a simulation model of shearing stress distribution within the brain following concussive injury, rotational acceleration produces maximum shearing forces mainly distributed in the center of the brain, also known as the peri-thalamic region. These forces may cause thalamocortical disruptions, which could last longer even than cortical dysfunction. The thalamus is a centrally located relay station with nerve fibers projecting out to the cerebral cortex in all directions for information transmission. Thalamocortical interactions include both temporal synchronization between specific thalamic subnuclei and corresponding cortical regions and desynchronization between irrelevant areas. If such interactions are out of sync, multiple neuropsychiatric symptoms, such as neuropathic pain, tinnitus, Parkinson's disease, schizophrenia epilepsy, and depression, may arise.

In addition, the thalamus has also been implicated to be related to the development and recovery of PCSs. With its global long-range connections, the thalamus plays a role in the neuroplasticity mechanism in the injured brain and can facilitate the improvement of PCSs. Patients with multi-year coma after traumatic brain injury exhibited significant improvement after thalamic stimulation. In other words, the thalamic injuries may potentially lead to the failure in postconcussive neuroplasticity mechanism, resulting in the prolonged PCSs. Despite the importance of thalamocortical interaction, and the negative impacts of thalamic injury on consciousness and cognition, little is known about the mechanism that sets off such chain of events that consequently lead to multiple neurological and psychiatric symptoms.

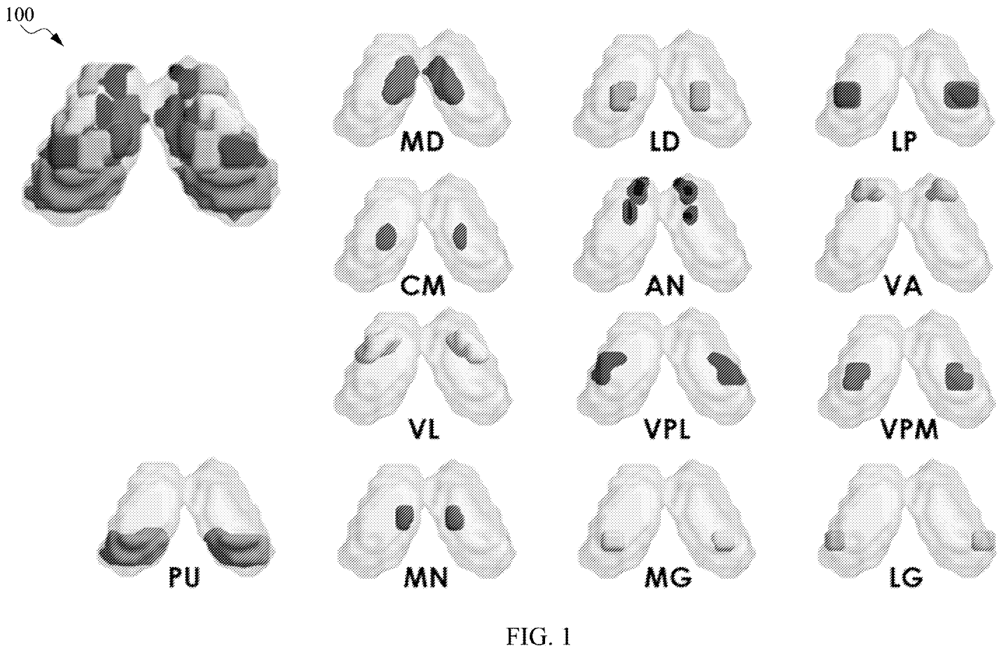

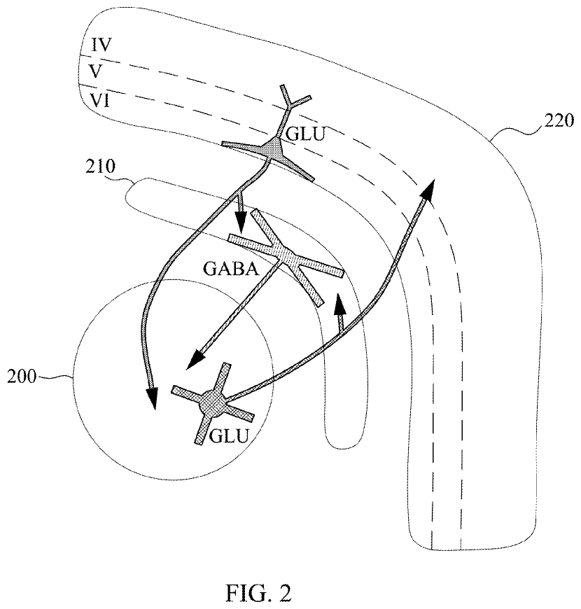

FIG. 1 is a perspective view of a thalamus and nuclear groups therein in accordance with an embodiment of the present disclosure.

A thalamus 100 may act as a regulatory center that integrates memory, cognitive, and somatosensory information. The thalamus 100 may include a plurality of thalamic nuclear groups (or thalamic sub-nuclei), such as medical dorsal nucleus (MD), lateral dorsal (LD), lateral posterior (LP), centromedian (CM), anterior nuclear group (AN), ventral anterior nucleus (VA), ventral lateral nucleus (VL), ventral posterolateral nucleus (VPL), ventral posteromedial nucleus (VPM), pulvinar (PU), medial geniculate nucleus (MG), and lateral geniculate nucleus (LG), as shown in FIG. 1.

FIG. 2 is a diagram showing the thalamic reticular nucleus (TRN) with respect to the thalamus and cortex in accordance with an embodiment of the present disclosure.

The thalamic reticular nucleus (TRN) 210 is a sheet of GABergic (Gamma-Aminobutyric Acid ergic) cells situated along the rostral and lateral surface of the dorsal thalamus. The thalamic reticular nucleus is the only GABAergic thalamic subdivision that provides recurrent inhibition of thalamocortical relay cells, and has been proposed to serve as the “brain pacemaker” that modulates thalamocortical synchrony. As shown in FIG. 2, the thalamic reticular nucleus 210 may be situated between the thalamus 200 and the neocortex 220. Since the TRN 210 is a very thin layer located at the junction of the thalamus and brain tissue, it is extremely sensitive to shear stress. Glutamatergic corticothalamic neurons send projections back to the TRN 210 and other thalamic nuclei (e.g., the neocortex 220). Roman numerals of the neocortex 220 indicate cortical layers. In FIG.2, “GABA” represents “gamma-amino butyric acid”; and “GLU” represents “glutamate.” For example, on the basis of a simulation model of shearing stress within the brain following mTBI (mild traumatic brain injury), rotational acceleration produced maximum shearing stress around the center core of the brain (i.e., peri-thalamic region).

Since the thalamus contributes to a broad range of critical functions, the side effects of thalamic and/or thalamocortical damage can vary from person to person. We first hypothesized that 1) the extent of concussion-induced thalamic damage and the disturbed thalamocortical coherence should be quantifiable, and be able to explain not only clinical between-group differences (e.g., patient vs. healthy control [HC]), but also within-group variances (e.g., symptom-prolonged patients versus symptom-resolved patients); 2) as thalamocortical coherence is restored, so should one's PCSs. To this end, we longitudinally recruited 70 patients with concussion and 70 demographically-matched healthy controls (HCs), and followed the patients for 2 years on their neuroimaging and neuropsychological data.

For example, patients with concussion (defined by the following criteria: closed-head injury (CHI), loss of consciousness lasting <30 minutes, initial Glasgow Coma Scale score>13, and normal head CT results) and demographically matched healthy controls (HCs) were recruited from Taipei Medical University Hospital (TMUH) between September 2015 and September 2021 (i.e., shown in Table 1). Their baseline assessments were collected within the first two weeks of injury, and follow-ups were conducted at 1 and 2 years after mTBI.

Exclusion criteria were any history of prior brain injury, coexisting or previous neurological or psychiatric illness, and contraindication for MRI. Exclusion criteria for the HCs were the same as the additional requirement of no history of concussion or brain injury.

In some embodiments, Glasgow Outcome Scale-Extended (GOSE) and Mini-Mental State Examination (MMSE) were administered by a clinical psychologist during baseline assessment. Neuropsychological assessments, including Pittsburgh sleep quality index (PSQI), Rivermead Post Concussion Symptoms Questionnaire (PCSQ), Beck Anxiety Inventory (BAI), and Beck Depression Inventory (BDI), were administered on the same day as the initial and follow-up scans.

In some embodiments, the MRI data of the patients and HCs was obtained using a 3T (Tesla) MR (magnetic resonance) scanner (Siemens MAGNETOM Prisma, Erlangen, Germany) equipped with a 20-channel head coil. In addition, diffusion tensor imaging (DTI) was performed through echo-planar imaging (EPI) with two b values (1000 and 2500 s/mm²) along 64 noncollinear and noncoplanar diffusion directions and 10 b0 images. Standard single-shot gradient-echo EPI-based fMRI was also performed. The participants were requested to keep their eyes closed and not engage in any thoughts while remaining awake, alert, and as motionless as possible. For the coregistration and normalization of fMRI and DTI data, three-dimensional T₁-weighted magnetization-prepared rapid gradient-echo images were obtained.

In some embodiments, the DTI data were preprocessed using FSL 5.0.10 (FMRIB, Oxford, UK) and MRtrix 3.0.2 (Brain Research Institute, Melbourne, Australia). Specifically, denoising, Gibbs ringing artifact removal, and bias field correction were applied using MRtrix and then processed according to the standard FSL pipeline (i.e., brain mask estimation along with eddy-current and motion correction). In addition, Fixel-based analysis of fiber density was performed using the MRtrix multishell and multitissue constrained spherical deconvolution method and the probabilistic streamlining method. DTI-derived parametric maps, including fractional anisotropy (FA), radial diffusivity (RD), and track density (TD) maps, were generated and then spatially normalized to the template space by using MRtrix.

In some embodiments, the fMRI data were preprocessed using Statistical Parametric Mapping (SPM12; Wellcome Department, University College London, UK) for slice timing correction, realignment, spatial normalization to template space, and spatial smoothing with a Gaussian kernel. Linear and quadratic trends of the fMRI time series were also removed.

In some embodiments, the human cortical regions of interest were defined using Brodmann area (BA) and the thalamic sub-nuclei were delineated using the Talairach atlas. The six head-motion parameters and averaged white matter and cerebrospinal fluid signals were partially regressed out of the preprocessed fMRI time series. The within-thalamic functional connectivity was estimated by calculating the Pearson correlation coefficients between the temporal filtered (0.01-0.08 Hz) fMRI time series, followed by Fisher Z transform. The magnitude-squared thalamocortical functional coherence was calculated using in-house MATLAB (version R2020a; Mathworks, Sherborn, MA, USA) scripts. The highly idiosyncratic nature of concussion led to differences in each patient's collision angle, position, and structural damage. To compensate for this variation, thalamocortical coherence was first calculated between the averaged time series of the voxels from the entire thalami and each cortical region so as to determine the disease-dominant frequency range that most accurately reflected the disturbed thalamocortical coherence.

In some embodiments, the connectome matrix was composed of within-thalamic functional connectivity and thalamocortical coherence estimated from the temporal filtered fMRI time series between each thalamic sub-nucleus and cortical region in the disease-dominant frequency range.

In some embodiments, a one-sample t test was employed to determine the within-group significance in the HC and mTBI groups, and two-tailed two-sample t testing was used to determine between-group differences. A two-tailed paired-sample t test was applied to examine the significant differences between the initial and follow-up data. Pearson correlation coefficients were computed to reveal the relationships between functional coherence and neuropsychological or behavioral changes. The statistical tests were corrected by controlling the false discovery rate (FDR) at q=0.01 to prevent errors related to multiple comparisons.

In some embodiments, the connectome biomarkers obtained through baseline measurement along with patient's age and sex were treated as possible features to train a machine learning-based predictive model. For example, the machine learning-based predictive model may be a support vector machine regressor, a convolutional neural network (CNN), or a deep neural network (DNN), but the present disclosure is not limited thereto.

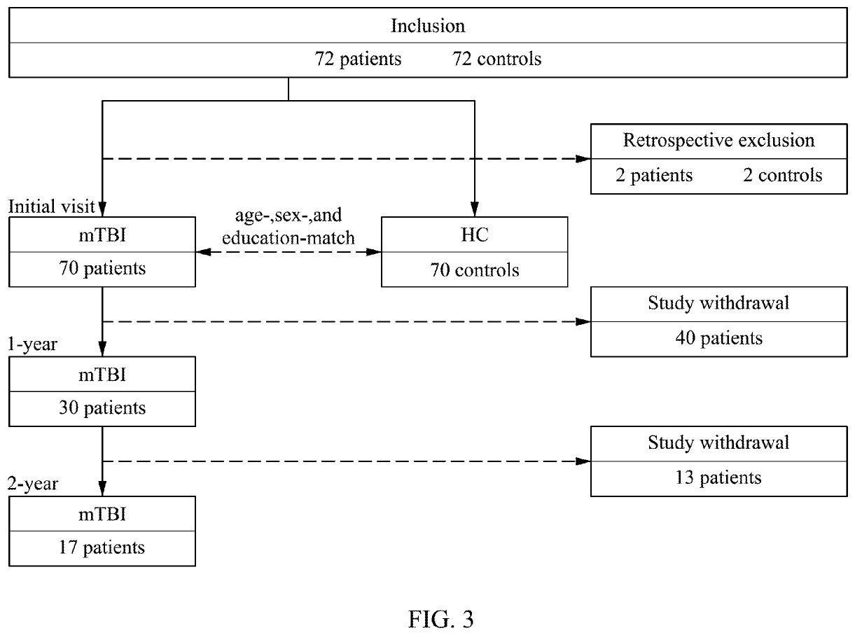

FIG. 3 is a flowchart of participant recruitment in accordance with an embodiment of the present disclosure.

In an embodiment, 70 patients with mTBI (age=37.9±12.2 y; 47 [67.1%] female) and 70 demographically matched HCs (age=36.7±12.0 y; 47 [67.1%] female) were recruited. One patient and two healthy controls (HCs) were retrospectively excluded because of severe MRI artifacts due to dental prosthesis, and another patient was excluded due to pituitary gland tumor diagnosis. The injury mechanisms were as follows: motor vehicle accident (n=37), fall (n=17), sports (n=3), assault (n=8), and other (n=5). The main reasons for patient dropout in the follow-up visits included unresponsiveness to phone calls or emails and change in address or job.

The follow-up rates at 1 and 2 years for the patients were 42.9% (n=30) and 24.3% (n=17), respectively. The demographic characteristics, clinical characteristics, and cognitive characteristics and working memory task performance of the enrolled individuals are shown in Table 1 as follows:

TABLE 1

| | | Patients With | Patients With | Patients With |
|---|---|---|---|---|
| Demographic | | mTBI | mTBI | mTBI |
| Characteristics | HCs | (Baseline) | (1-y Follow-Up) | (2-y Follow-Up) |
| Sample size (n) | 70 | 70 | 30 | 17 |
| Sex (F/M) | 47/23 | 47/23 | 22/8 | 9/8 |
| Age (y) | 36.66 ± 12.04 | 37.97 ± 12.24 | 36.80 ± 11.29 | 35.76 ± 10.97 |
| Education (y) | 15.54 ± 3.98 | 14.45 ± 3.03 | 14.65 ± 2.76 | 14.71 ± 2.82 |
| GOSE score | N.A. | 6.20 ± 1.98 | 6.10 ± 2.11 | 5.63 ± 2.16 |
| MMSE score | 29.47 ± 0.74 | 28.44 ± 1.61 | 28.80 ± 1.45 | 28.47 ± 1.59 |
| Postinjury days | N.A. | 6.76 ± 2.85 | 5.83 ± 3.15 | 5.29 ± 2.69 |
| (initial scan; d) | | | | |
| Postinjury days | N.A. | N.A. | 373.83 ± 14.60 | 739.59 ± 17.08 |
| (follow-up; d) | | | | |

Clinical Characteristics

| | | | | |
|---|---|---|---|---|
| PSQI | 4.86 ± 2.20 | 8.07 ± 4.36 | 6.67 ± 9.45 | 7.35 ± 4.05 |
| PCSQ score | 4.94 ± 5.22 | 13.30 ± 13.43 | 9.03 ± 9.45 | 9.29 ± 11.66 |
| BAI score | 3.91 ± 4.32 | 9.80 ± 9.57 | 7.27 ± 7.84 | 7.43 ± 8.06 |
| BDI score | 4.56 ± 4.61 | 9.70 ± 9.86 | 6.97 ± 6.18 | 7.93 ± 6.57 |

The data in Table 1 is presented as means±standard deviations. The MMSE score, PSQI, PCSQ score, BAI score, and BDI score differed significantly between patients with mTBI and HCs. These symptoms did not improve significantly at the 1-year follow-up. The PSQI, PCSQ score, and BDI score did not improve significantly, even after 2 years of mTBI. The abbreviations MMSE, PSQI, PCSQ, BAI, and BDI may refer to Mini-Mental State Examination, Pittsburgh sleep quality index, Rivermead Post Concussion Symptoms Questionnaire, Beck Anxiety Inventory, Beck Depression Inventory. Specifically, the neuropsychological measures, such as MMSE, PSQI, PCSQ, BAI, and BDI scores, can be used to quantitatively measure the neuropsychological state of each of the patients and HCs.

In addition, 19 and 10 of all patients had persistent symptoms at the 1-year and 2-year follow-up, as shown by Tables 2-1 and 2-2:

TABLE 2-1

| | | | mTBI Patients |
|---|---|---|---|
| | | | With Symptoms |
| Demographic | | Patients With | Resolved |
| Characteristics | HCs | mTBI at Baseline | Within 1 y |
| Sample size (n) | 70 | 70 | 11 |
| Sex (female/male) | 47/23 | 47/23 | 5/6 |
| Age (y) | 36.66 ± 12.04 | 37.97 ± 12.24 | 28.91 ± 7.20 |
| Education (y) | 15.54 ± 3.98 | 14.45 ± 3.03 | 15.45 ± 1.75 |
| GOSE score | N.A. | 6.20 ± 1.98 | 6.80 ± 1.81 |
| MMSE score | 29.47 ± 0.74 | 28.44 ± 1.61 | 29.00 ± 1.26 |
| Postinjury days | N.A. | 6.76 ± 2.85 | 6.18 ± 2.96 |
| (initial scan; d) | | | |
| Postinjury days | N.A. | N.A. | 374.82 ± 15.75 |
| (follow-up; d) | | | |

Clinical Characteristics

| | | | |
|---|---|---|---|
| PSQI | 4.86 ± 2.20 | 8.07 ± 4.36 | 5.27 ± 3.44 |
| PCSQ score | 4.94 ± 5.22 | 13.30 ± 13.43 | 0.91 ± 0.94 |
| BAI score | 3.91 ± 4.32 | 9.80 ± 9.57 | 4.09 ± 4.18 |
| BDI score | 4.56 ± 4.61 | 9.70 ± 9.86 | 3.73 ± 4.96 |

TABLE 2-2

| | mTBI Patients With | mTBI Patients With | mTBI Patients With |
|---|---|---|---|
| Demographic | Symptoms Prolonged | Symptoms Resolved | Symptoms Prolonged |
| Characteristics | Over 1 y | Within 2 y | Over 2 y |
| Sample size (n) | 19 | 7 | 10 |
| Sex (female/male) | 17/2 | 1/6 | 8/2 |
| Age (y) | 41.37 ± 10.79 | 32.33 ± 10.39 | 38.60 ± 11.42 |
| Education (y) | 14.18 ± 3.16 | 15.33 ± 2.34 | 14.20 ± 3.22 |
| GOSE score | 5.74 ± 2.21 | 6.40 ± 2.30 | 5.50 ± 2.07 |
| MMSE score | 28.47 ± 1.59 | 29.17 ± 1.33 | 28.10 ± 1.73 |
| Postinjury days | 5.63 ± 3.32 | 5.00 ± 1.73 | 5.10 ± 3.14 |
| (initial scan; d) | | | |
| Postinjury days | 373.26 ± 14.31 | 751.6 ± 26.25 | 735.50 ± 9.65 |
| (follow-up; d) | | | |

Clinical Characteristics

| | | | |
|---|---|---|---|
| PSQI | 7.47 ± 3.42 | 5.67 ± 4.55 | 8.60 ± 3.66 |
| PCSQ score | 13.74 ± 8.93 | 1.00 ± 0.89 | 15.20 ± 12.12 |
| BAI score | 9.11 ± 8.92 | 1.50 ± 3.00 | 10.89 ± 8.02 |
| BDI score | 8.84 ± 6.14 | 3.50 ± 5.74 | 10.87 ± 5.56 |

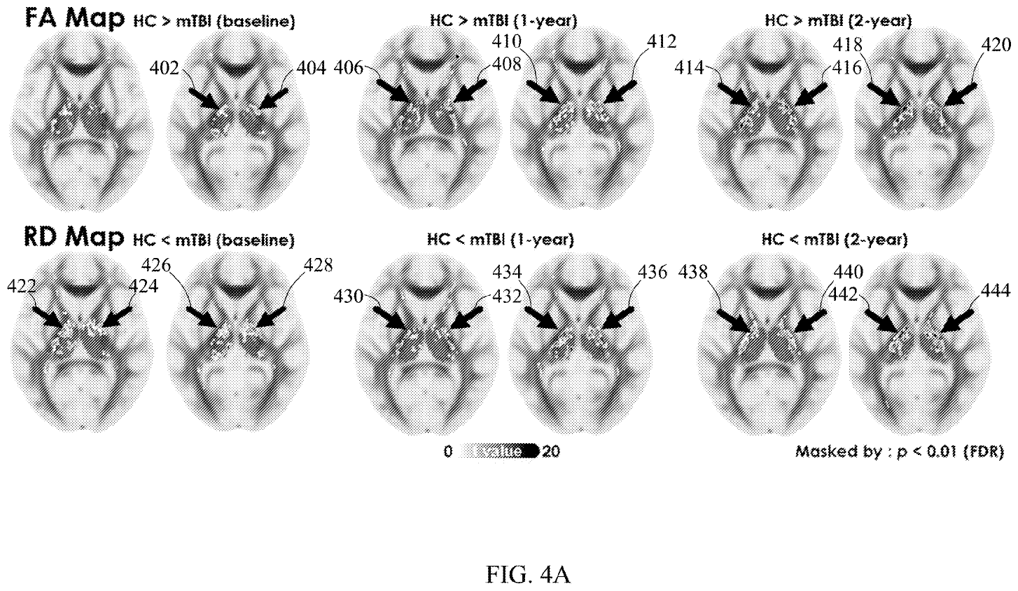

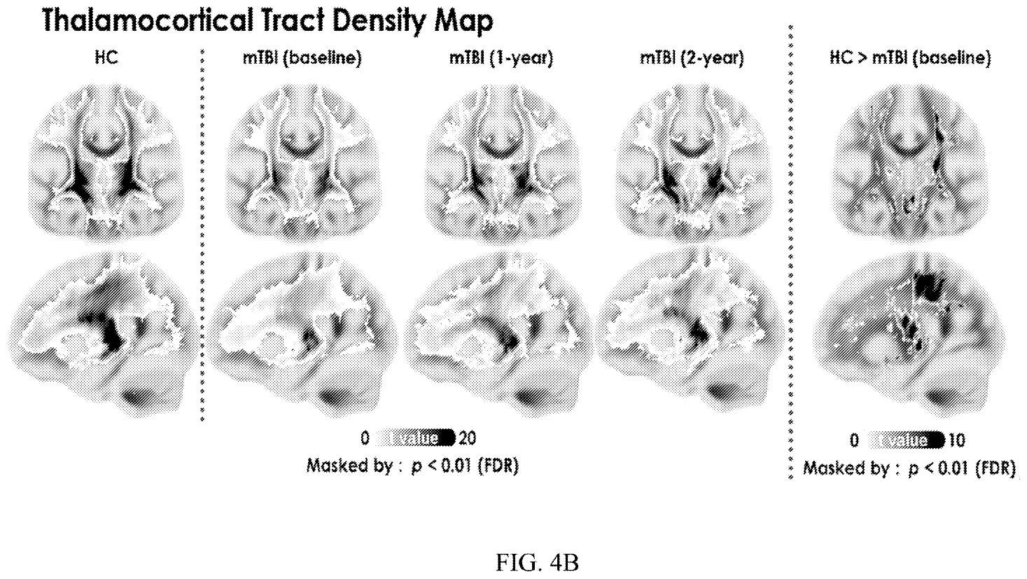

FIGS. 4A-4B are diagrams illustrating thalamocortical tracts injury and peri-thalamic injury centered around the thalamic reticular nucleus (TRN) on DTI in accordance with the embodiment of FIG. 3. Please refer to FIG. 3 and FIGS. 4A-4B.

The upper portion and lower portion of FIG. 4A may respectively include fractional anisotropy (FA) maps and radial diffusivity (RD) maps in patients compared with HCs at baseline as well as at 1-year and 2-year follow-ups. Translucent dark ovals indicate locations of the bilateral thalami. These maps were further masked by the threshold of the group-averaged FA (>0.2) to ensure that only the white matter tracts remained.

In an embodiment, at the boundaries of the bilateral thalami, where thalamic reticular nucleus (TRN) was located, a significant increase in radial diffusivity (RD) and decrease in fractional anisotropy (FA) in patients compared with HCs at baseline as well as at 1-year and 2-year follow-ups, as shown by arrows 402-444 in FIG. 4A. These may indicate long-lasting axonal damage, myelin damage, or both in the peri-thalamic TRN after concussion. It should be noted that bilateral destruction of the rostral pole of GABAergic thalamic reticular nucleus (TRN) may promote thalamocortical dysrhythmia (probably by dis-inhibition). In addition, the TRN injury may be remained identifiable for up to 2 years after concussion.

FIG. 4B shows the thalamocortical tract density (TD) map in patients compared with HCs at baseline as well as at 1-year and 2-year follow-ups. The top row in FIG. 4B may present the coronal slice passing through the center of the bilateral thalami, and the bottom row in FIG. 4B may indicate the sagittal slice passing through the center of the left thalamus. In addition, a significant decrease was noted in the white matter tract density (TD) of patients at baseline, suggesting the presence of white matter tract damage, as shown in FIG. 4B. This tract damage was resolved within 1 year after concussion, indicating that tract damage is temporary and reversible compared with the long-lasting TRN damage. For example, the white matter tract microstructural damage may indicate impaired thalamocortical tract. The tract injury may be negligible after 1 or 2 years, probably because of repair. However, the TRN injury may last longer than tract injury, and the protracted TRN injury may be the main contributor of prolonged PCSs.

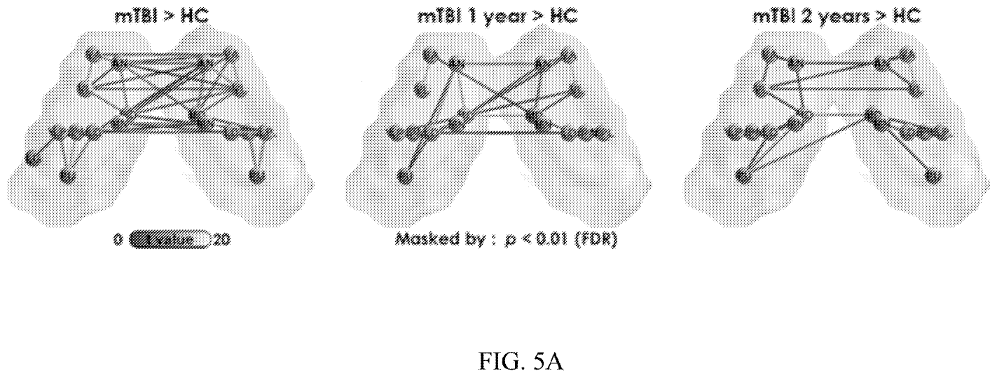

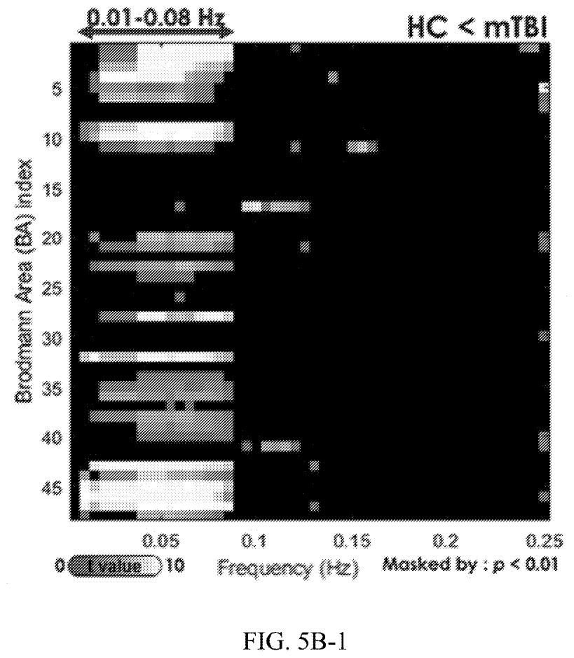

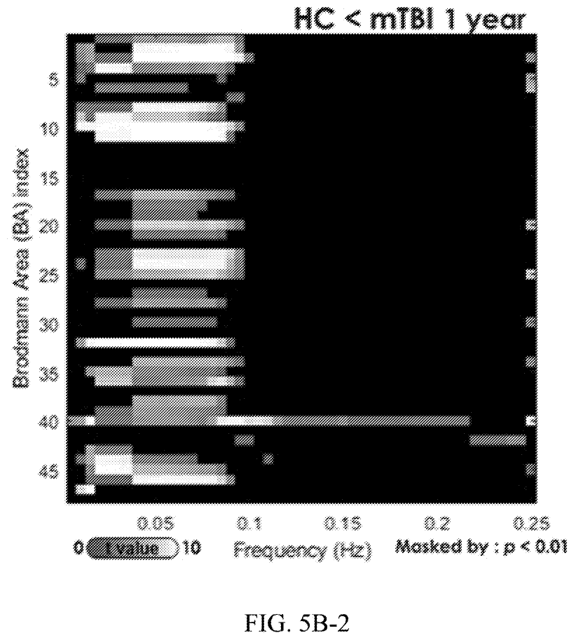

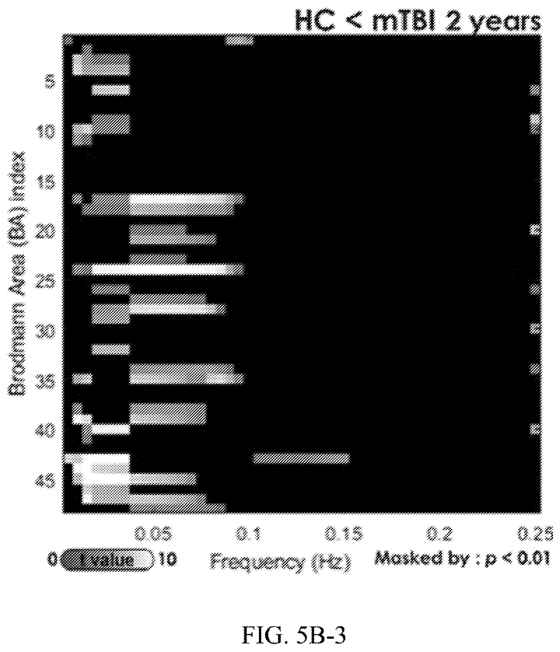

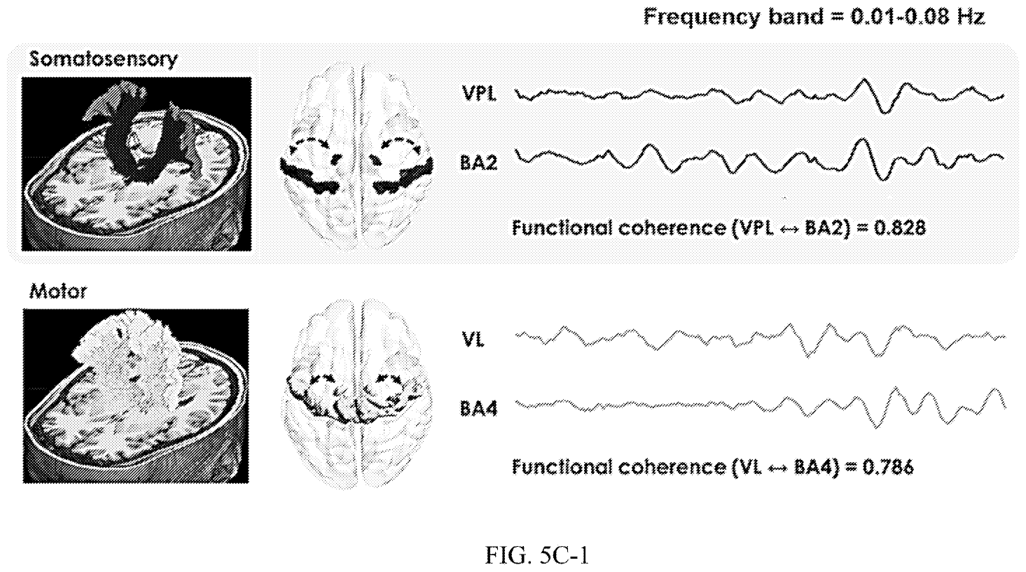

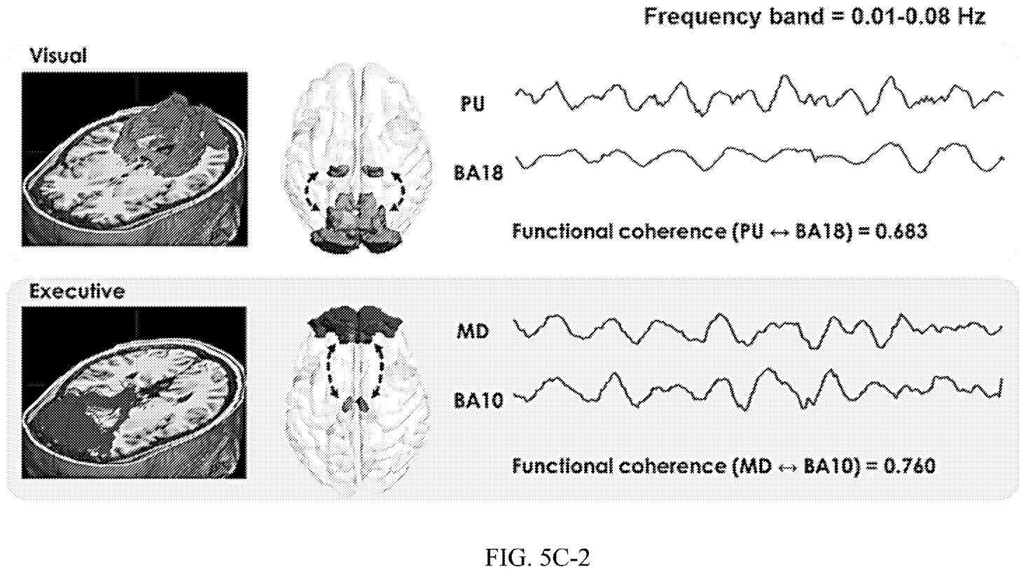

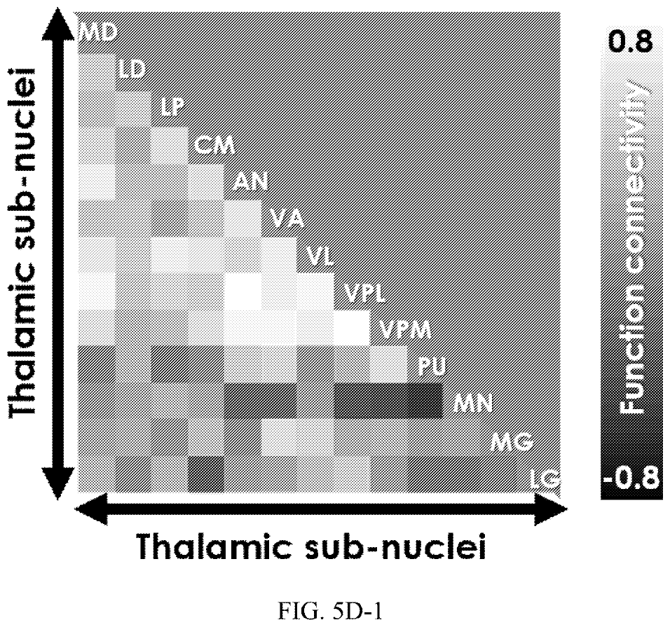

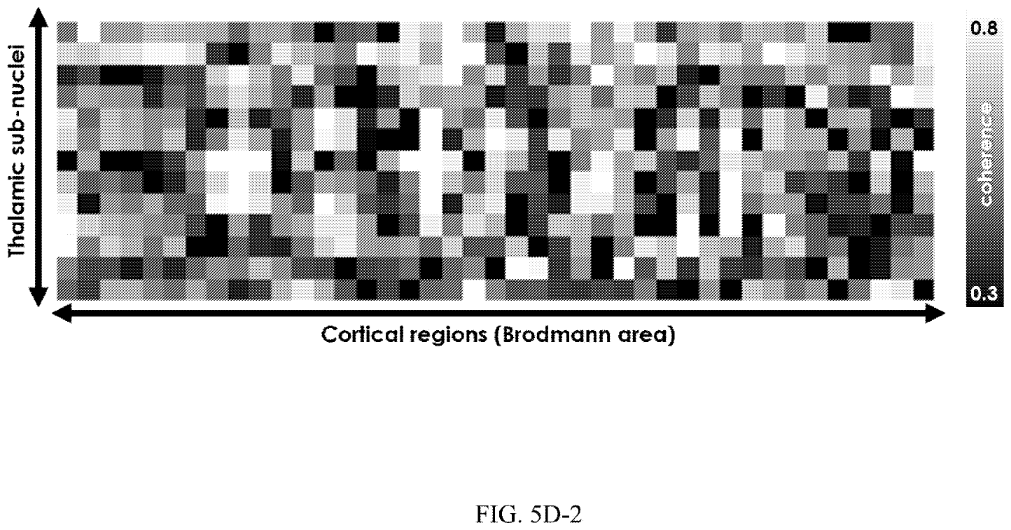

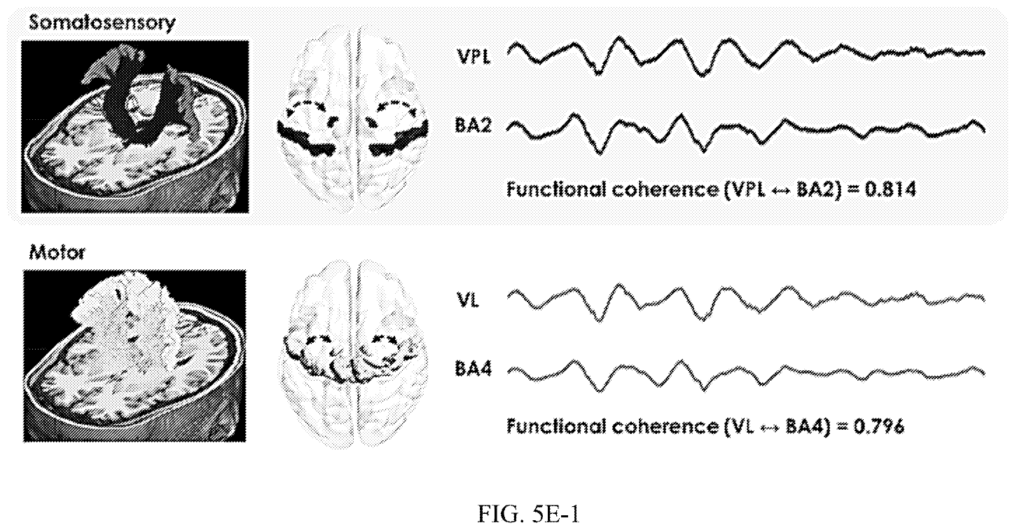

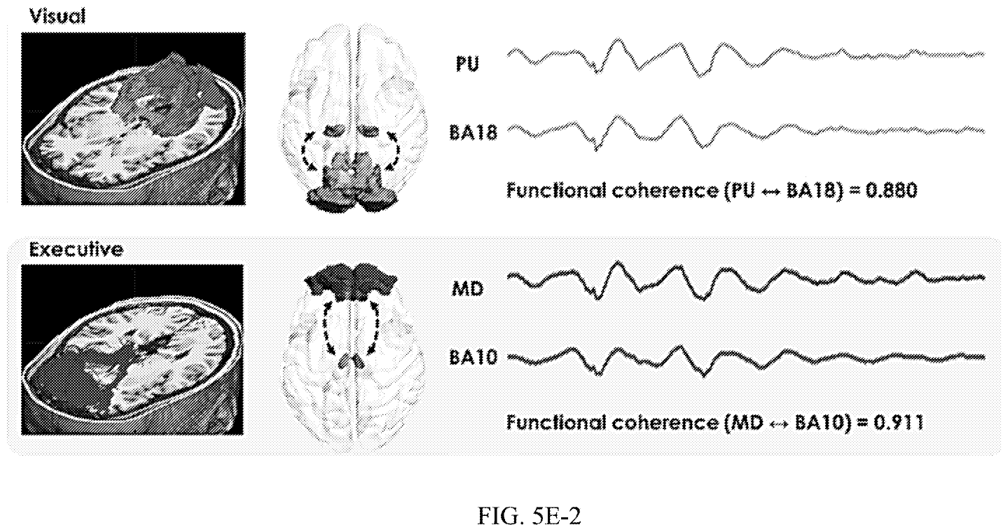

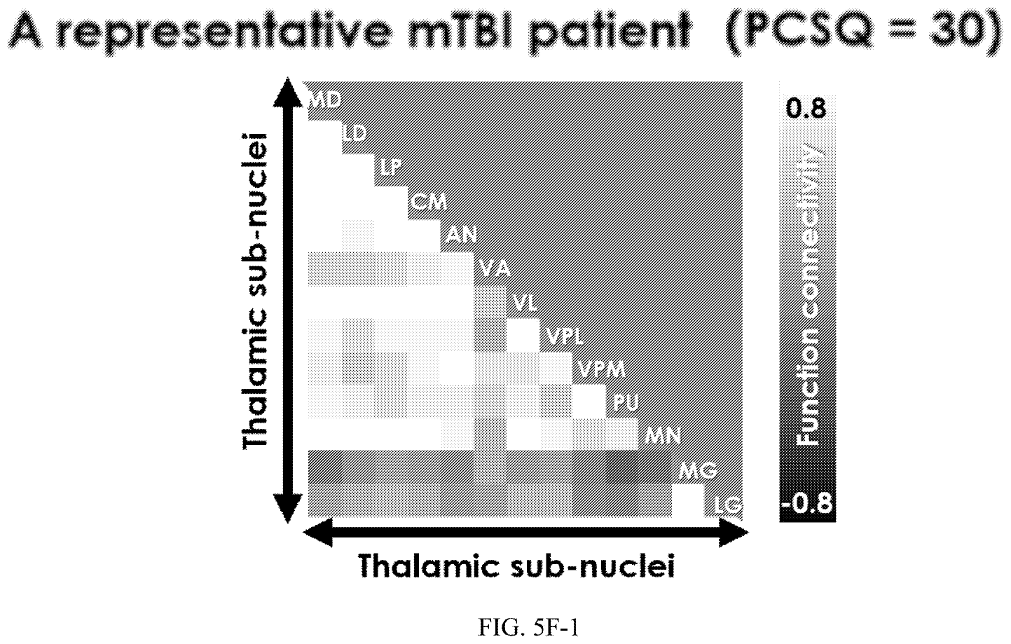

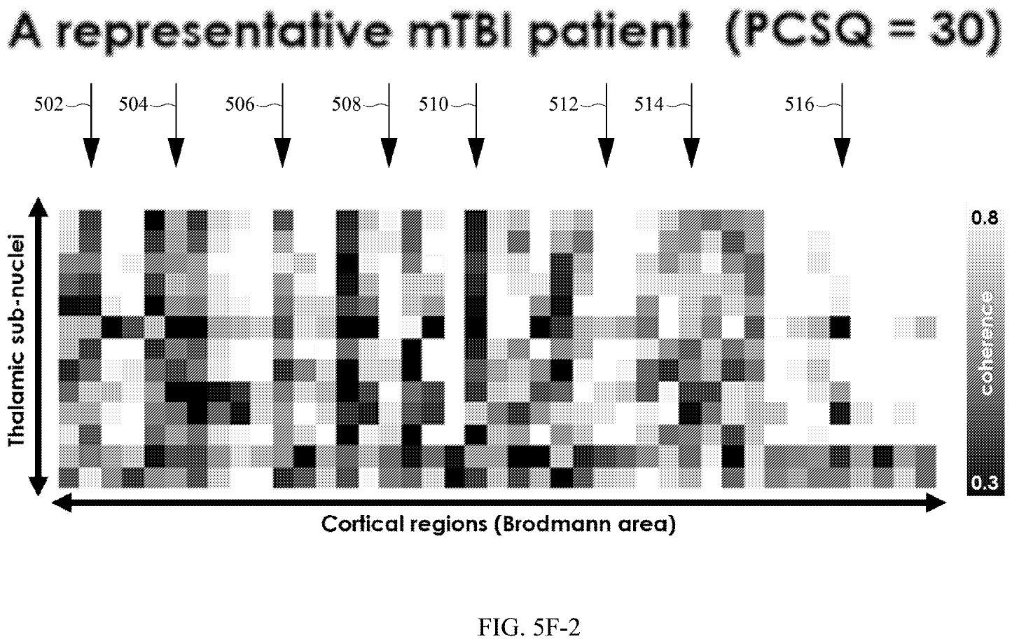

FIG. 5A is a diagram illustrating significantly increased within-thalamic resting-state functional connectivity in patients compared with the HCs in baseline assessment and follow-ups in accordance with an embodiment of the present disclosure. FIGS. 5B-1 to 5B-3 are diagrams illustrating increase in coherence between thalami and almost all cortical regions in the low-frequency band in patients compared with HCs in baseline assessment and follow-ups in accordance with the embodiment of FIG. 5A.

In an embodiment, patients exhibited resting state hyperconnectivity among their thalamic subdivisions in patients (i.e., shown in FIG. 5A). In other words, the rs-fMRI (resting-state functional magnetic resonance imaging) signal in different thalamic subdivisions became similar (i.e., higher functional coherence) after concussion, as shown in FIGS. 5E-5F, which may imply the failure of thalamic function in relaying and integrating the different information. This significant elevation in within-thalamic functional connectivity was also observed at the 1-year and 2-year follow-ups in patients after concussion (i.e., shown in FIG. 5A), indicating no significant restoration of the disrupted thalamic function in patients.

FIGS. 5C-1 and 5C-2 are diagrams illustrating rs-fMRI signals in different thalamic subdivisions for a representative HC in accordance with the embodiment of FIG. 5A. FIGS. 5D-1 and 5D-2 are diagrams illustrating within-thalamic functional connectivity matrix and thalamocortical coherence matrix for a representative HC in accordance with the embodiment of FIG. 5A. FIGS. 5E-1 and 5E-2 are diagrams illustrating rs-fMRI signals in different thalamic subdivisions for a representative patient with mTBI in accordance with the embodiment of FIG. 5A. FIGS. 5F-1 and 5F-2 are diagrams illustrating within-thalamic functional connectivity matrix and thalamocortical coherence matrix for a representative patient with mild traumatic brain injury (mTBI) in accordance with the embodiment of FIG. 5A.

In an embodiment, the rs-fMRI signals in different thalamic subdivisions and their corresponding cortical regions for a representative HC is shown in FIGS. 5C-1 and 5C-2. For purposes of description, the rs-fMRI signals of the thalamic subdivisions of VPL, VL, PU, and MD and their corresponding cortical regions (e.g., Brodmann Area: BA2, BA4, BA18, and BA10) are shown in FIGS. 5C-1 and 5C-2. It should be noted that there are also many other thalamic subdivisions in the thalamus, and the details can be referred to in the embodiment of FIG. 1. In addition, the within-thalamic functional connectivity matrix and thalamocortical coherence matrix for a representative HC are shown in the left portion and right portion of FIGS. 5D-1 and 5D-2, respectively.

For example, the coherence between the rs-fMRI signals of a specific thalamic subdivision and a specific cortical region can be computed using formula (1) as follows:

$${C}_{x y} ( f ) = \frac{{\vert {G}_{x y} ( f ) \vert}^{2}}{{G}_{xx} ( f ) {G}_{y y} ( f )} \qquad ( 1 )$$

where C_{xy}(f) denotes the coherence between two signals x(t) and y(t); G_{xy}(f) denotes the cross-spectral density between x and y; and Gₓₓ(f) and G_{yy}(f) denote the auto spectral density of x and y, respectively. The magnitude of the spectral density is denoted as |G|. The coherence value C_{xy}(f) will be between 0 and 1.

The within-thalamic functional connectivity matrices shown in FIGS. 5D-1 and 5F-1 may illustrate the coherence between the rs-fMRI signals of two different thalamic subdivisions. For purposes of illustration, if the rs-fMRI signals of the two thalamic subdivisions are very similar, the gray scale of the corresponding block (i.e., element) will be higher (i.e., lighter). If the rs-fMRI signals of the two subdivisions are not similar, the gray scale of the corresponding block will be lower (i.e., darker). As can be seen from FIG. 5D-1, only few parts of the thalamic subdivisions has a higher coherence for a representative HC.

In addition, the rs-fMRI signals in different thalamic subdivisions and their corresponding cortical regions for a representative patient with mTBI is shown in FIG. 5E. Similarly, the fMRI signals of the thalamic subdivisions of VPL, VL, PU, and MD and their corresponding cortical regions (e.g., Brodmann area: BA2, BA4, BA18, and BA10) are shown in FIGS. 5E-1 and 5E-2. It should be noted that there are also many other thalamic subdivisions in the thalamus, and the details can be referred to in the embodiment of FIG. 1. In addition, the within-thalamic functional connectivity matrix and thalamocortical coherence matrix for a representative patient with mTBI are shown in the left portion and right portion of FIGS. 5F-1 and 5F-2, respectively.

In comparison with FIGS. 5C-1 and 5C-2, the fMRI signals in different thalamic subdivisions and their corresponding cortical regions for a representative patient with mTBI shown in FIGS. 5E-1 and 5E-2 may substantially have higher functional coherence. In addition, most of the blocks in the within-thalamic functional connectivity matrix shown in FIG. 5F-1 may have relatively high coherence values (e.g., close to 1 or −1). In addition, arrows 502 to 516 in FIG. 5F may show the abnormal patterns of pathologically elevated low-frequency thalamocortical rhythmicity, showing a high degree of temporal coherence with almost all cortical regions. It should be noted that the rs-fMRI signals were band-pass filtered to 0.01 to 0.08 Hz before generating the within-thalamic functional connectivity matrix and thalamocortical coherence matrix in FIG. 5D and FIG. 5F.

In some embodiments, because of the highly idiosyncratic nature of concussions, the concussion-induced thalamic and/or thalamocortical damage can vary from person to person. All elements in the within-thalamic functional connectivity matrices shown in FIGS. 5D-1 and 5F-1 may be averaged as a compromise strategy to individually quantify the degree of elevation in within-thalamic functional connectivity and yield the potential personalized disease burden of thalamic dysfunction. Notably, the averaged within-thalamic connectivity at baseline was significantly correlated with patient PCSQ scores and BDI scores. In some other embodiments, the whole within-thalamic functional connectivity matrices (i.e., no averaging is applied) can be used to quantify the degree of elevation in within-thalamic functional connectivity.

As shown by the thalamocortical functional coherence matrices in of FIGS. 5D-2 and 5F-2, an increase in rhythmic coherence between the thalamus and almost all cortical regions in the frequency range=0.01-0.08 Hz (disease-dominant frequency range; p<0.01 [FDR]). Specifically, the globally enhanced thalamocortical coherence interfered with the cortical regions in the different thalamocortical loops (e.g., arrows 502 to 516 in FIG. 5F-2) indicated the rs-fMRI signals between different thalamocortical loops became similar after concussion, thus disrupted the thalamocortical multifunctionality.

In some embodiments, all elements in the thalamocortical coherence matrix was averaged as a compromise strategy to individually quantify the degree of pathologically increased thalamocortical coherence. In some other embodiments, the whole thalamocortical functional coherence matrix (i.e., no averaging is applied) can be used to quantify the degree of pathologically increased thalamocortical coherence.

### Roles of TRN Injury and Disrupted Thalamocortical Coherence in PCS Prolongation

### Clinical Human Results

Please refer to Table 1 and Table 2. Table 1 presents the PCSs progression estimated via PSQI, PCSQ scores, and BDI scores of the patients with mTBI and the HCs. Table 2 further divides the symptom-resolved and symptom-prolonged patients according to a published cut-off score (PCSQ≥3). In an embodiment, the number of patients with mTBI at 1-year follow-up is 30, and they can be divided by “symptom-resolved” and “symptom-prolonged” groups using a published cut-off score (PCSQ>3), where the number of patients in the symptom-resolve group (i.e., PCSQ<3) is 11, and the number of patients in the symptom-prolonged group (i.e., PCSQ>3) is 19. The symptom-prolonged group may have significantly lower fractional anisotropy (FA) and higher radial diffusivity (RD) scores near the peri-thalamic TRN, higher within-thalamic functional connectivity, and enhanced thalamocortical coherence in comparison with the symptom-resolved group, implying that the level of acute TRN injury and the altered thalamocortical coherence may be related to the prolongation of PCSs.

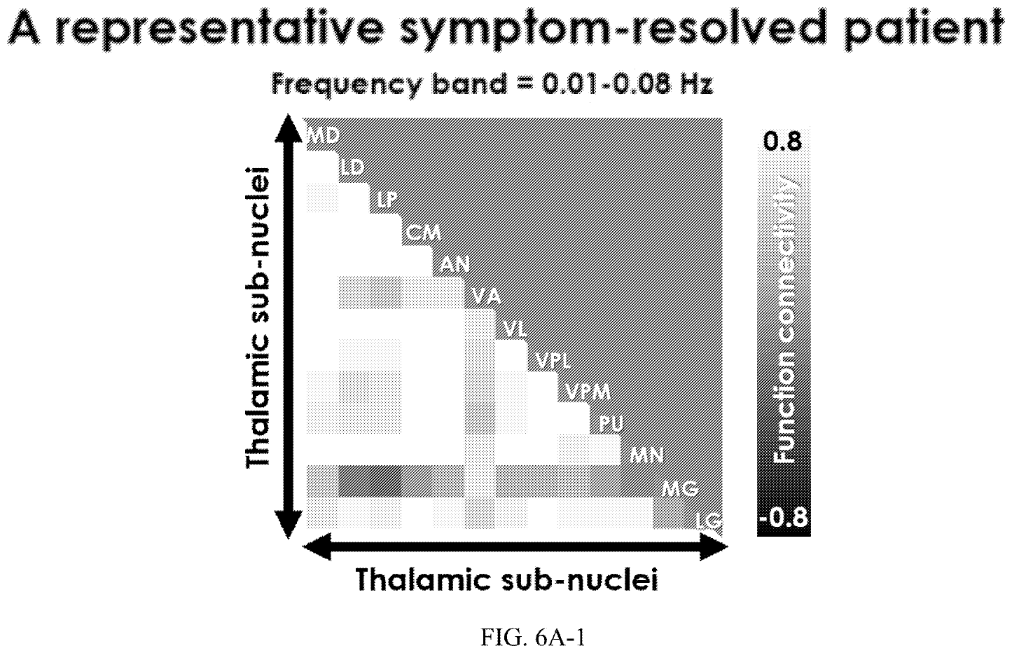

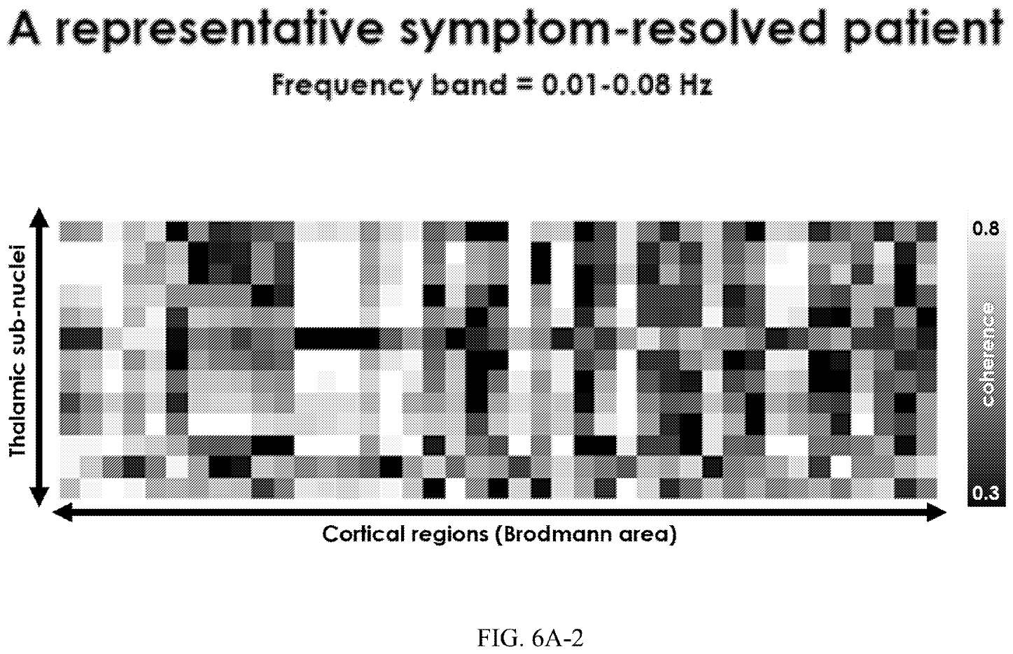

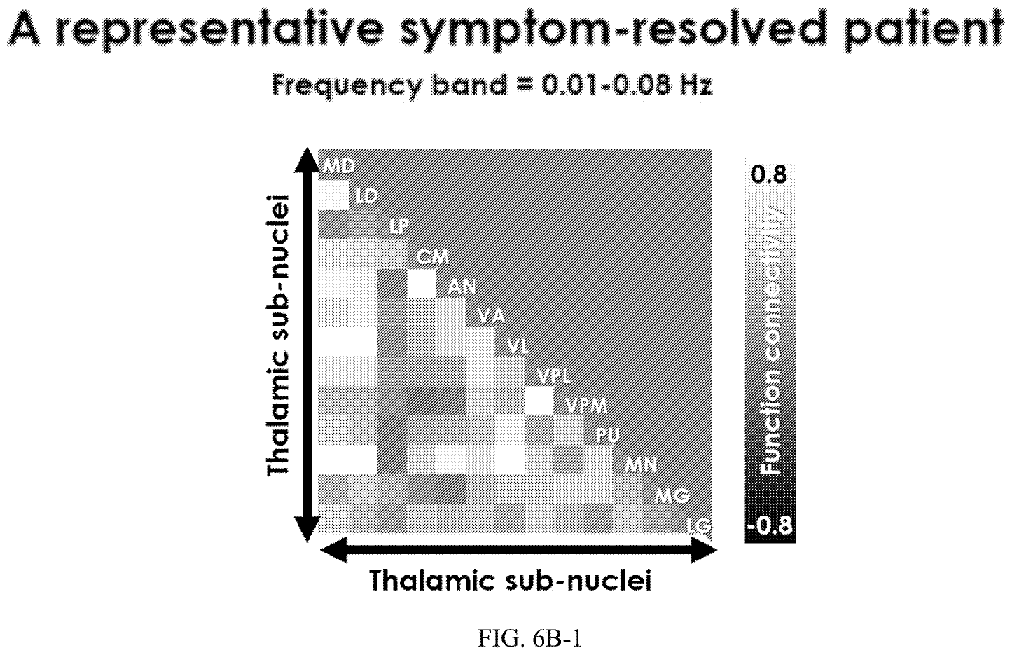

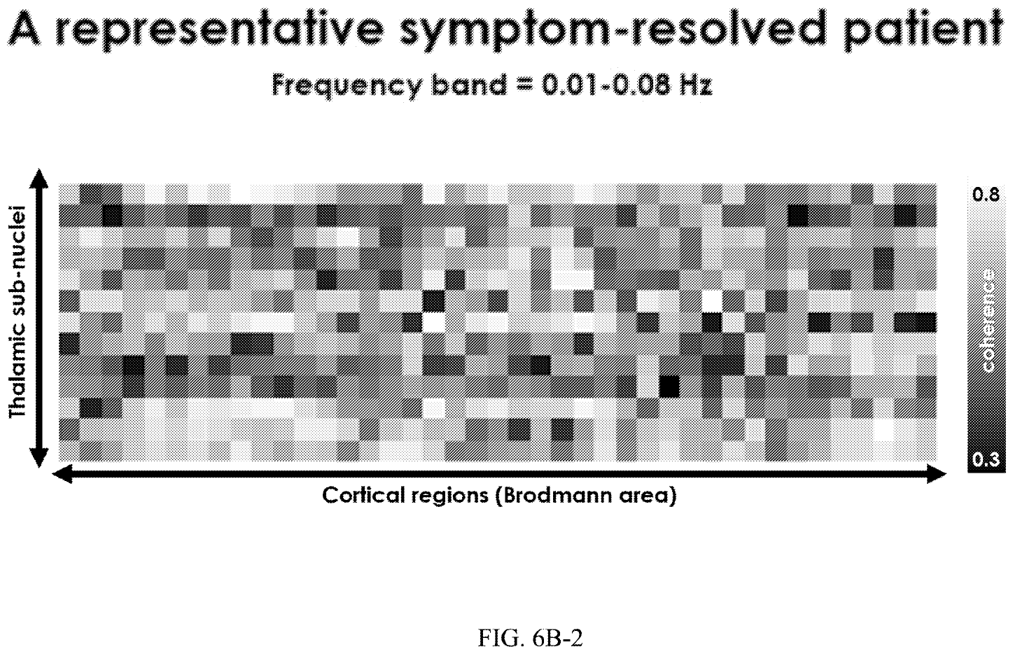

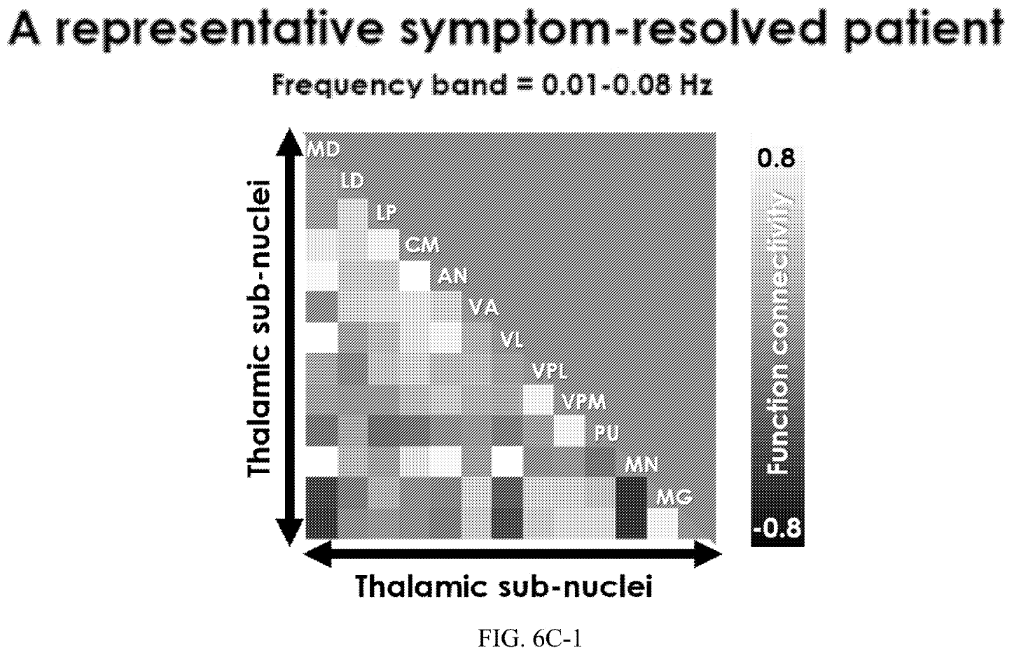

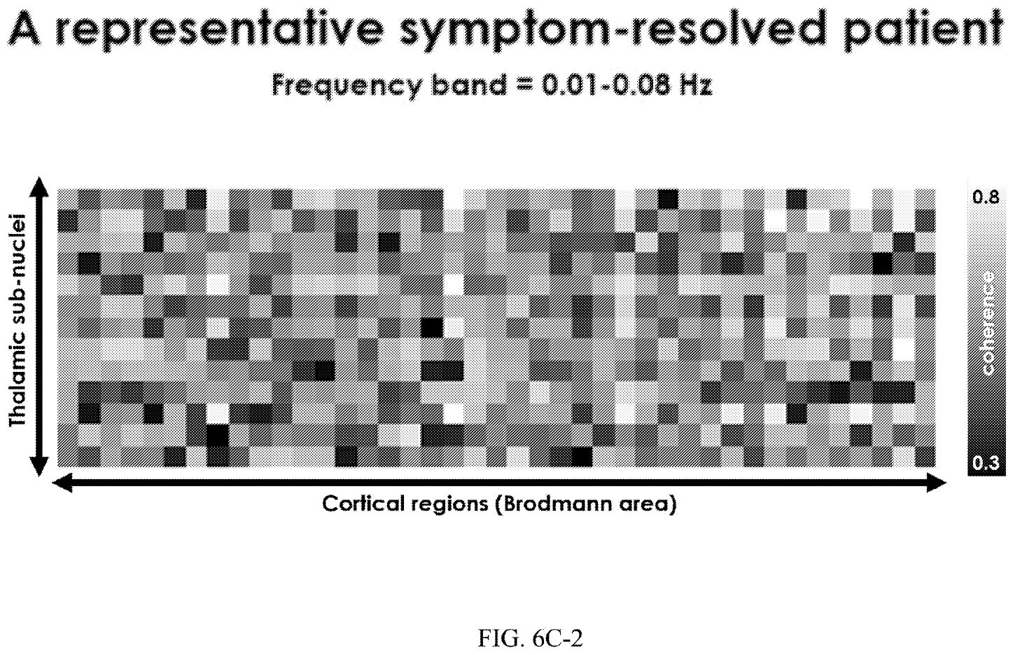

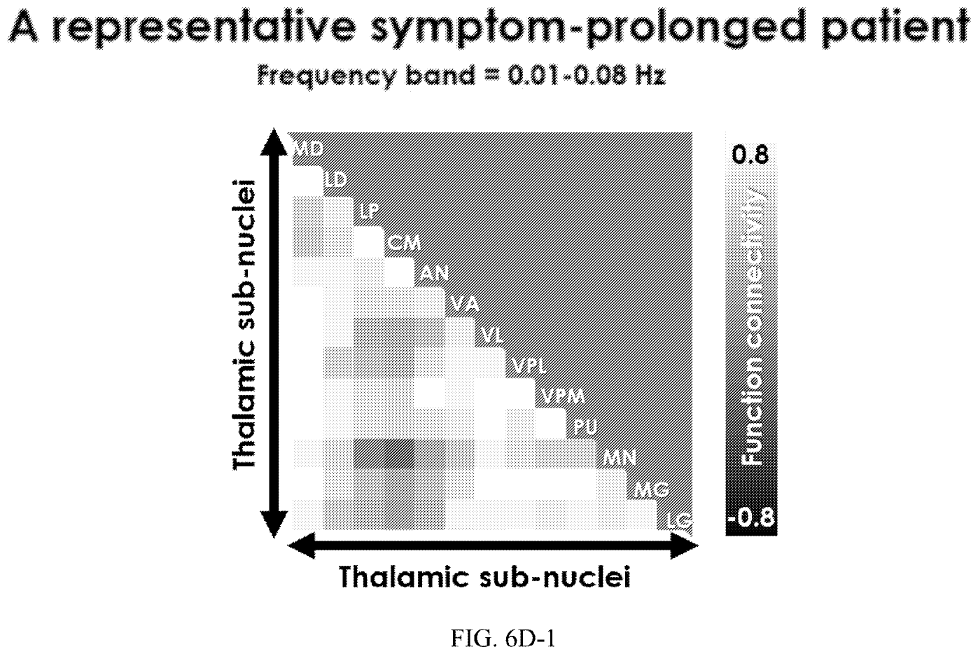

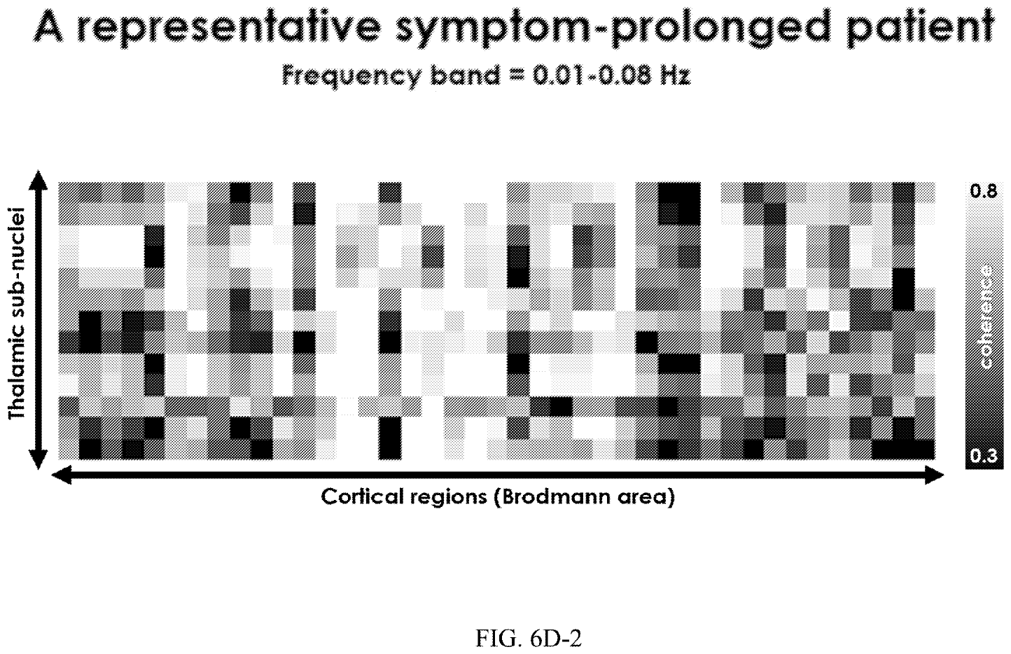

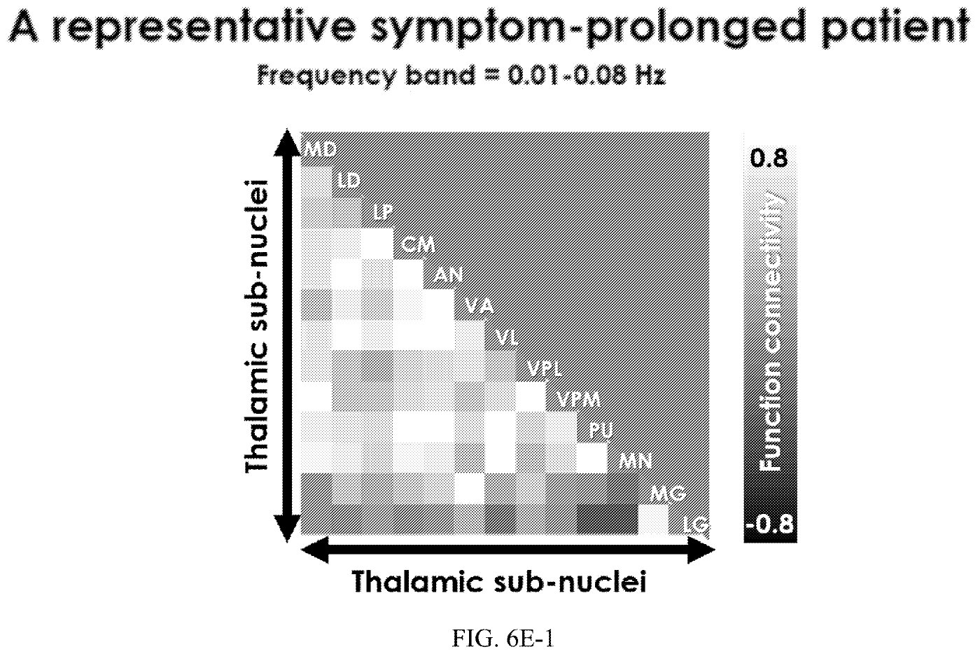

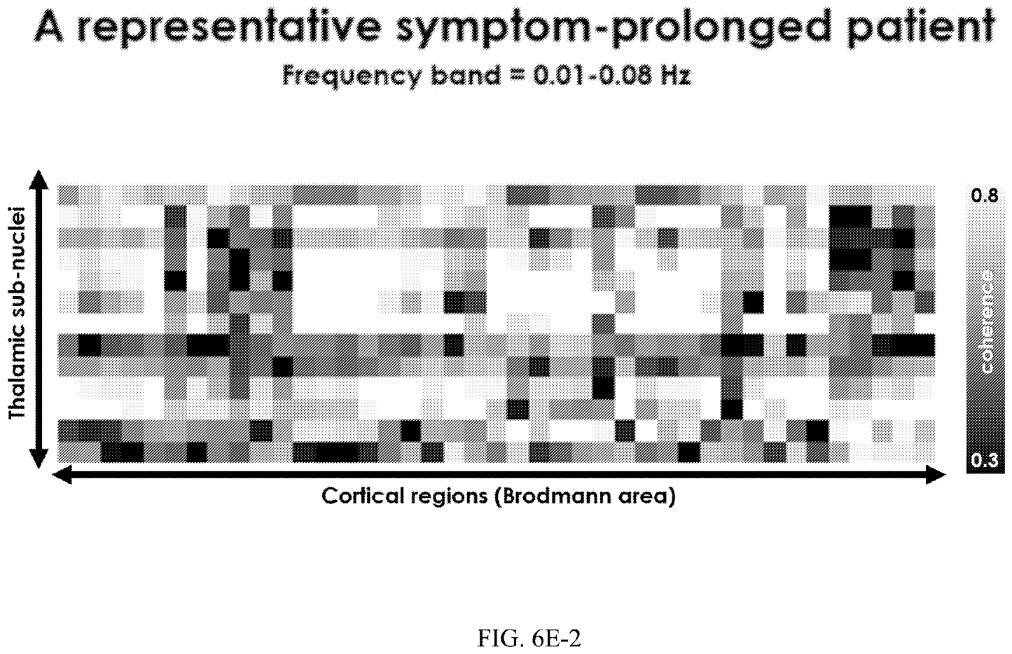

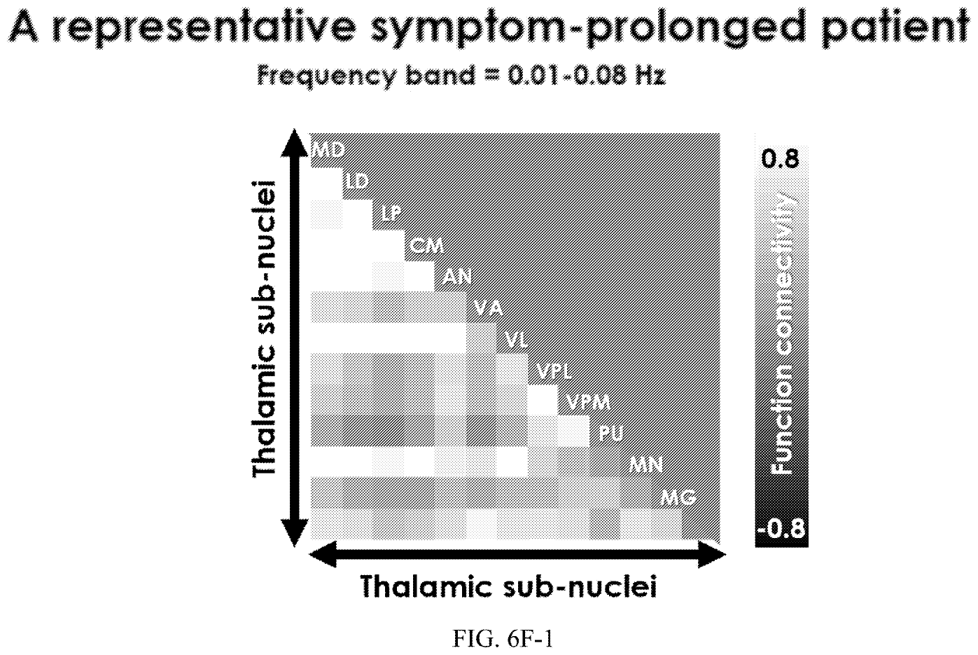

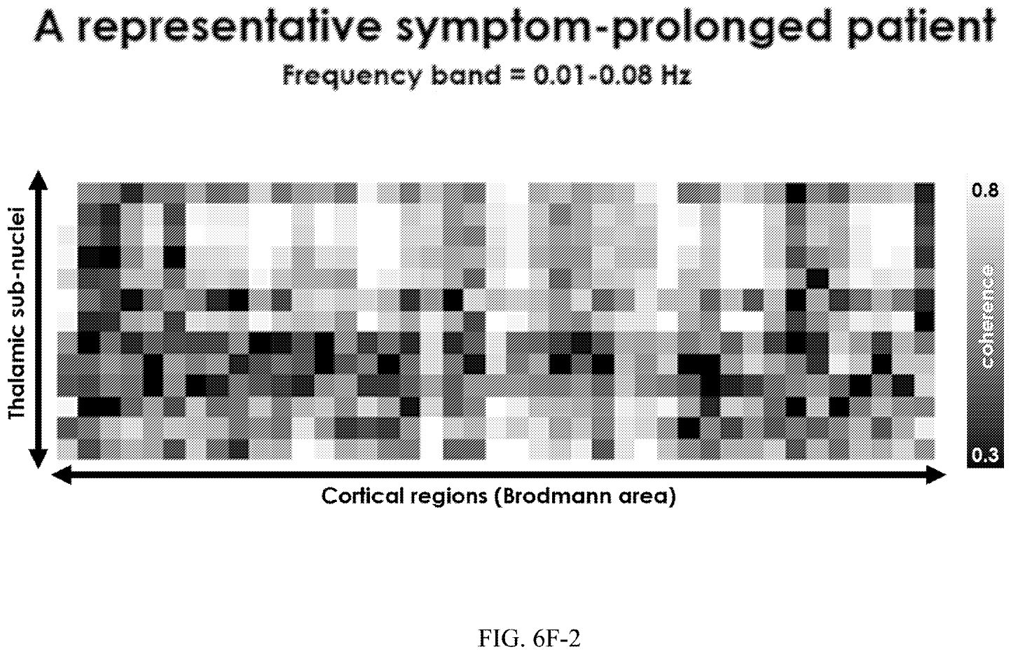

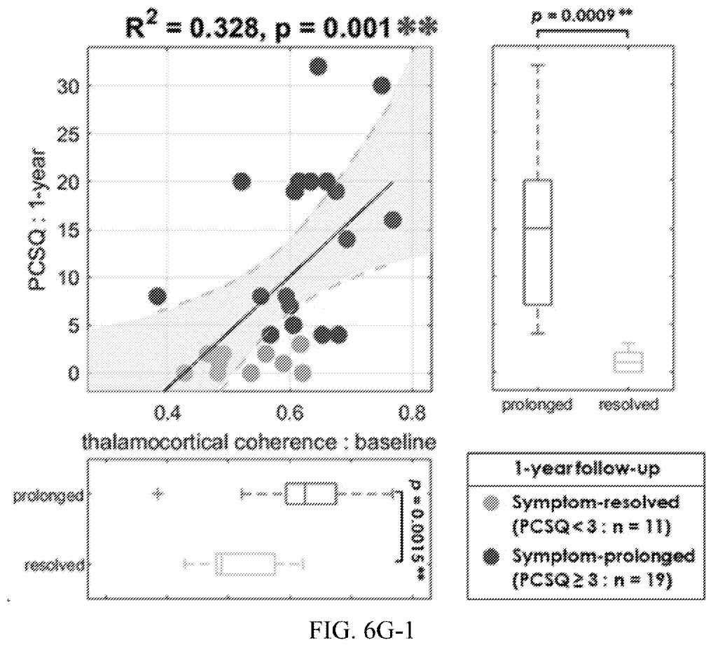

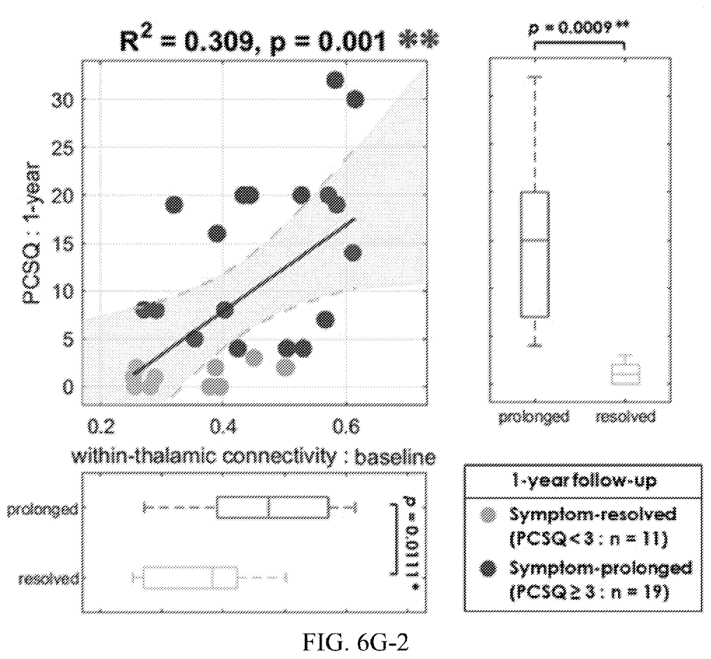

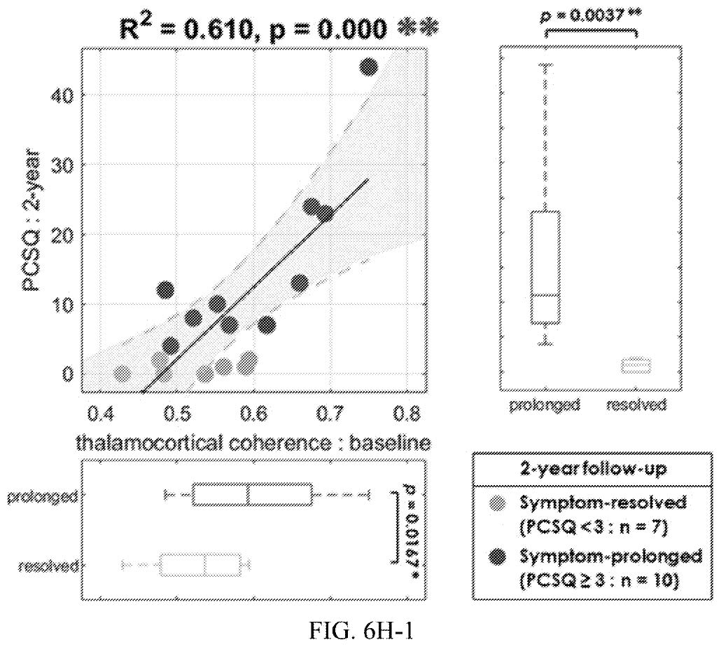

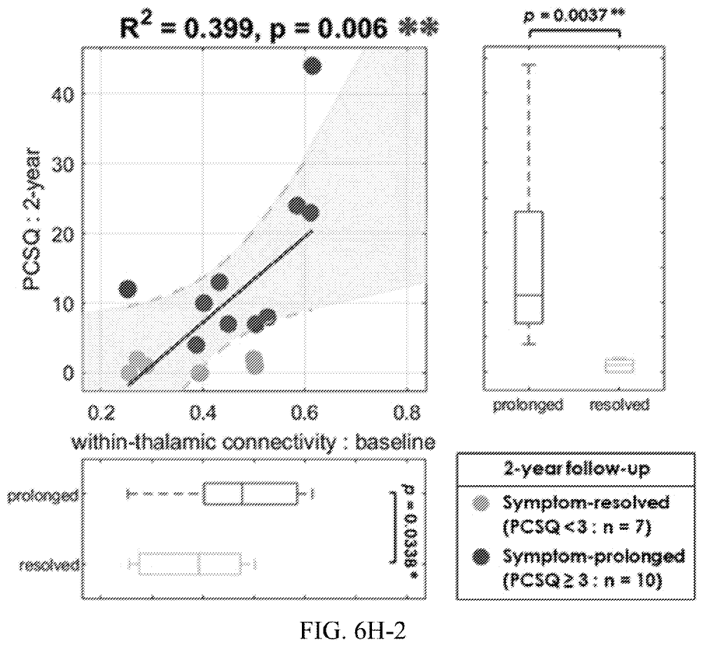

For example, FIGS. 6A-1, 6B-1, and 6C-1 illustrate the within-thalamic functional connectivity matrices for a representative symptom-resolved patient at baseline, 1-year, and 2-year assessment, respectively. FIGS. 6A-2, 6B-2, and 6C-2 illustrate the thalamocortical functional coherence matrix for a representative symptom-resolved patient at baseline, 1-year, and 2-year assessment, respectively.

FIGS. 6D-1, 6E-1, and 6F-1 illustrate the within-thalamic functional connectivity matrix (left) for a representative symptom-prolonged patient at baseline, 1-year, and 2-year assessment, respectively. FIGS. 6D-2, 6E-2, and 6F-2 illustrate the thalamocortical functional coherence matrix for a representative symptom-prolonged patient at baseline, 1-year, and 2-year assessment, respectively. The rs-fMRI signals were band-pass filtered to 0.01 to 0.08 Hz before calculating functional connectivity and coherence in FIGS. 6A-6F. The long-term PCSs after 1 year (i.e., shown in FIGS. 6G-1 and 6G-2) and 2 years (shown in FIGS. 6H-1 and 6H-2) are correlated with thalamocortical dysrhythmia (TCD)-related connectome biomarkers, including the averaged low-frequency thalamocortical coherence, and averaged within-thalamic hyperconnectivity. Dots with a dark color indicate symptom-prolonged patients (Rivermead Post Concussion Symptoms Questionnaire [PCSQ] score≥3); Dots with a lighter color represent symptom-resolved patients (PCSQ score<3).

At both 1-year and 2-year follow-ups, the symptom-resolved and symptom-prolonged patients exhibited distinct patterns of baseline biomarkers (i.e., the within-thalamic functional connectivity and thalamocortical coherence); both of which were strongly associated with the degree of long-term symptoms, as shown in FIGS. 6A-6H. These pathological patterns in the thalamocortical coherence matrix not only aided in dichotomizing patients with prolonged and resolved symptoms (e.g., shown in FIGS. 6A-6F) but also served to correlate with the trajectory of the symptom progression in patients with prolonged symptoms and resolved symptoms (e.g., as shown in of FIGS. 6B, 6C, 6E, and 6F), suggesting that as thalamocortical coherence is restored, so should one's PCSs.

Specifically, the two connectome biomarkers, such as the within-thalamic functional connectivity matrix and thalamocortical coherence matrix, of the patients with mTBI at baseline and the patients' age and sex in the Taipei Medical University Hospital (TMUH) cohort may be used as training data for the machine learning-based predictive model.

In some embodiments, the machine learning-based predictive model may be a support vector machine or a classifier, and the within-thalamic functional connectivity matrix and thalamocortical coherence matrix of a given patient with mTBI as baseline can be simplified as a first average value of all elements in the within-thalamic functional connectivity matrix and a second average value of the thalamocortical coherence matrix. The first average value and the second average value can be input to the machine learning-based predict model along with the age and sex of the given patient for training. Since the first average value and the second average value may correspond to a PCSQ score of the given patient, after the machine learning-based predictive model is trained using training data of a certain number of patients, the machine learning-based predictive model may predict a first PCSQ score and a second PCSQ score of the given patient at both 1-year and 2-year follow-ups. If the predicted first PCSQ score or the second PCSQ score is equal to or less than the predetermined PCSQ score (e.g., 3), the given patient will be predicted as a possible symptom-resolved patient in 1 year or 2 years. If the predicted first PCSQ score or the second PCSQ score is greater than the predetermined PCSQ score (e.g., 3), the given patient will be predicted as a possible symptom-prolonged patient in 1 year or 2 years.

In some other embodiments, the machine learning-based predictive model may be a convolutional neural network or a deep neural network, and the within-thalamic functional connectivity matrix and thalamocortical coherence matrix of a given patient with mTBI as baseline can be regarded as different two-dimensional arrays input to the machine learning-based predictive model along with the age and sex of the given patient for training. Similarly, since the first average value and the second average value may correspond to a PCSQ score of the given patient, after the machine learning-based predictive model is trained using training data (e.g., including the within-thalamic functional connectivity matrix, thalamocortical coherence matrix, age, and sex) of a certain number of patients, the machine learning-based predictive model may predict a first PCSQ score and a second PCSQ score of the given patient at both 1-year and 2-year follow-ups.

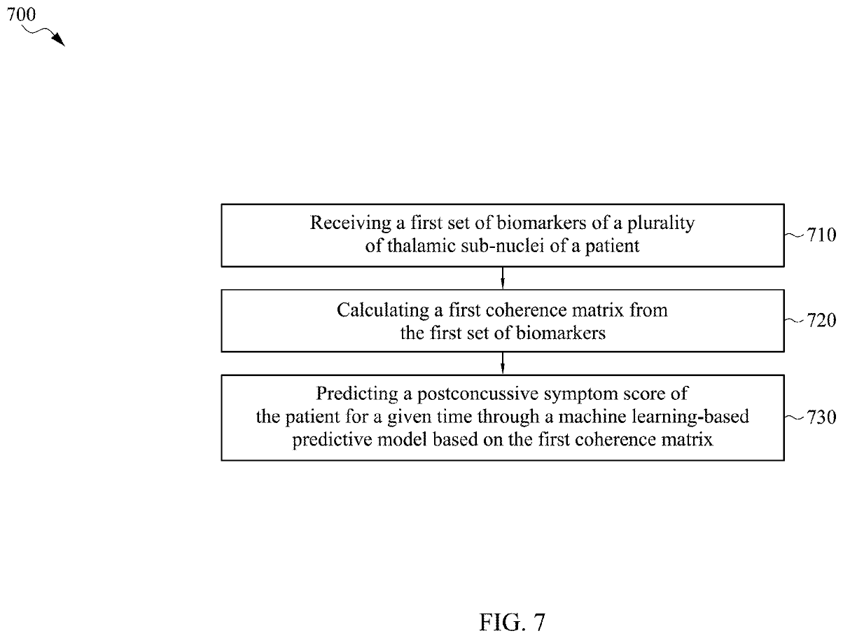

FIG. 7 is a flowchart of a method for predicting persistent postconcussive symptoms using thalamocortical coherence in accordance with an embodiment of the present disclosure.

In operation 710, a first set of biomarkers of a plurality of thalamic sub-nuclei of a patient is received. For example, the plurality of thalamic sub-nuclei may include medical dorsal nucleus (MD), lateral dorsal (LD), lateral posterior (LP), centromedian (CM), anterior nuclear group (AN), ventral anterior nucleus (VA), ventral lateral nucleus (VL), ventral posterolateral nucleus (VPL), ventral posteromedial nucleus (VPM), pulvinar (PU), medial geniculate nucleus (MG), and lateral geniculate nucleus (LG), as shown in FIG. 1. In addition, the first set of biomarkers may be rs-fMRI (resting-state functional magnetic resonance imaging) waveforms obtained by measuring each of the thalamic sub-nuclei of the patient.

In operation 720, a first coherence matrix (i.e., the within-thalamic functional connectivity matrix described above) is calculated from the first set of biomarkers. For example, each of the thalamic sub-nuclei has a corresponding biomarker (e.g., waveform), and the coherence between every two different thalamic sub-nuclei can be calculated using formula (1) described above. Thus, the first coherence matrix can be obtained by arranging the calculated coherences in a two-dimensional array.

In operation 730, a postconcussive symptom score of the patient for a given time is predicted through a machine learning-based predictive model based on the first coherence matrix. In some embodiments, the machine learning-based predictive model may be a support vector machine or a classifier, and the input of the machine learning-based predictive model may be a first average value of all elements in the first coherence matrix. In addition, the machine learning-based predictive model may output a postconcussive symptom score (e.g., a PCSQ score estimated by Rivermead postconcussive symptom questionnaire), the degree of which may indicate whether the symptom of the patient with mTBI will be resolved or prolonged in a later time (e.g., 1 year or 2 years later).

In some embodiments, a second set of biomarkers of a plurality of cortical regions of the patient is received. For example, the plurality of cortical regions may include 52 Brodmann areas (e.g., BA1 to BA52) which is defined and numbered by German anatomist Korbinian Brodmann in the early 1900's. In addition, the second set of biomarkers may be rs-fMRI (resting-state functional magnetic resonance imaging) waveforms obtained by measuring each of the cortical regions of the patient.

Afterwards, a second coherence matrix (i.e., the thalamocortical coherence matrix described above) is calculated from the second set of biomarkers. For example, each of the cortical regions has a corresponding biomarker (e.g., waveform), and the coherence between each thalamic sub-nucleus and each cerebral cortex region can be calculated using formula (1) described above. Thus, the second coherence matrix can be obtained by arranging the calculated coherences in a two-dimensional array. In some embodiments, the first coherence matrix and the second coherence matrix can be regarded as a first connectome matrix and a second connectome matrix, respectively.

In another embodiment, the postconcussive symptom score of the patient for a given time is predicted through a machine learning-based predictive model based on the first coherence matrix and the second coherence matrix. In yet another embodiment, information about the age and sex of the patient can be obtained, and the postconcussive symptom score of the patient for a given time is predicted through a machine learning-based predictive model based on the first coherence matrix, the second coherence matrix, and age and sex of the patient.

Specifically, the within-thalamic functional connectivity matrix and the thalamocortical coherence matrix of the patient as baseline can be derived from the first set of biomarkers of the thalamic sub-nuclei and the second set of biomarkers of the cortical regions, and they are shown to be related to the postconcussive symptom score (e.g., a PCSQ score estimated by Rivermead postconcussive symptom questionnaire) in the aforementioned embodiments. Thus, the within-thalamic functional connectivity matrix and the thalamocortical coherence matrix can be used to train the machine learning-based predictive model to predict the postconcussive symptom score of the patient for a given time (e.g., 1 year or 2 years later). Therefore, the method proposed in the present disclose can facilitate predicting the prolonged PCS of the patient using the within-thalamic functional connectivity matrix and the thalamocortical coherence matrix of the patient as baseline.

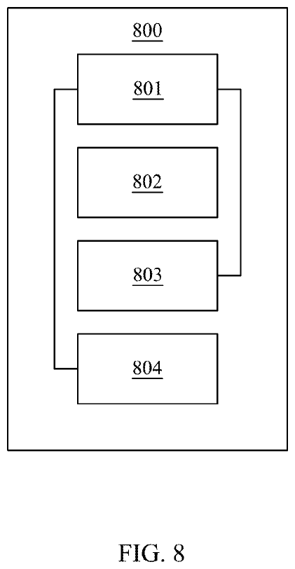

FIG. 8 is a schematic diagram showing a computer device 800 according to some embodiments of the present disclosure.

The computer device 800 may be capable of performing one or more procedures, operations, or methods of the present disclosure. The computer device 800 may be a server computer, a client computer, a personal computer (PC), a tablet PC, a set-top box (STB), a personal digital assistant (PDA), a cellular telephone, or a smartphone. The computing device 800 comprises processor 801, input/output interface 802, communication interface 803, and memory 804. The input/output interface 802 is coupled with the processor 801. The input/output interface 802 allows the user to manipulate the computing device 1100 to perform the procedures, operations, or methods of the present disclosure (e.g., the procedures, operations, or methods disclosed in FIGS. 4-7). The communication interface 803 is coupled with the processor 801. The communication interface 803 allows the computing device 800 to communicate with data outside the computing device 800, for example, receiving data including images or conditional features. A memory 804 may be a non-transitory computer readable storage medium. The memory 804 is coupled with the processor 801. The memory 804 has stored program instructions that can be executed by one or more processors (for example, the processor 801). In addition, the machine learning-based predictive model may be stored in the memory 804. Upon execution of the program instructions stored on the memory 804, the program instructions cause performance of the one or more procedures, operations, or methods disclosed in the present disclosure. For example, the program instructions may cause the computing device 800 to perform, for example, receiving a first set of biomarkers of a plurality of thalamic sub-nuclei of a patient; calculating a first coherence matrix from the first set of biomarkers; and predicting a postconcussive symptom score of the patient for a given time through a machine learning-based predictive model based on the first coherence matrix.

The scope of the present disclosure is not intended to be limited to the particular embodiments of the process, machine, manufacture, and composition of matter, means, methods, steps, and operations described in the specification. As those skilled in the art will readily appreciate from the disclosure of the present disclosure, processes, machines, manufacture, composition of matter, means, methods, steps, or operations presently existing or later to be developed that perform substantially the same function or achieve substantially the same result as the corresponding embodiments described herein may be utilized according to the present disclosure. Accordingly, the appended claims are intended to include within their scope processes, machines, manufacture, and compositions of matter, means, methods, steps, or operations. In addition, each claim constitutes a separate embodiment, and the combination of various claims and embodiments are within the scope of the disclosure.

The methods, processes, or operations according to embodiments of the present disclosure can also be implemented on a programmed processor. However, the controllers, flowcharts, and modules may also be implemented on a general purpose or special purpose computer, a programmed microprocessor or microcontroller and peripheral integrated circuit elements, an integrated circuit, a hardware electronic or logic circuit such as a discrete element circuit, a programmable logic device, or the like. In general, any device on which resides a finite state machine capable of implementing the flowcharts shown in the figures may be used to implement the processor functions of the present disclosure.

An alternative embodiment preferably implements the methods, processes, or operations according to embodiments of the present disclosure on a non-transitory, computer-readable storage medium storing computer programmable instructions. The instructions are preferably executed by computer-executable components preferably integrated with a network security system. The non-transitory, computer-readable storage medium may be stored on any suitable computer readable media such as RAMs, ROMs, flash memory, EEPROMs, optical storage devices (CD or DVD), hard drives, floppy drives, or any suitable device. The computer-executable component is preferably a processor, but the instructions may alternatively or additionally be executed by any suitable dedicated hardware device. For example, an embodiment of the present disclosure provides a non-transitory, computer-readable storage medium having computer programmable instructions stored therein.

While the present disclosure has been described with specific embodiments thereof, it is evident that many alternatives, modifications, and variations may be apparent to those skilled in the art. For example, various components of the embodiments may be interchanged, added, or substituted in the other embodiments. Also, all of the elements of each figure are not necessary for operation of the disclosed embodiments. For example, one of ordinary skill in the art of the disclosed embodiments would be able to make and use the teachings of the present disclosure by simply employing the elements of the independent claims. Accordingly, embodiments of the present disclosure as set forth herein are intended to be illustrative, not limiting. Various changes may be made without departing from the spirit and scope of the present disclosure.

Even though numerous characteristics and advantages of the present disclosure have been set forth in the foregoing description, together with details of the structure and function of the invention, the disclosure is illustrative only. Changes may be made to details, especially in matters of shape, size, and arrangement of parts, within the principles of the invention to the full extent indicated by the broad general meaning of the terms in which the appended claims are expressed.

What is claimed is:

1. A method for predicting persistent post-concussive neuropsychiatric symptoms based on thalamocortical coherence, the method comprising:
receiving a first set of biomarkers of a plurality of thalamic sub-nuclei of a patient;
calculating a first coherence matrix from the first set of biomarkers; and
predicting a postconcussive symptom score of the patient for a given time through a machine learning-based predictive model based on the first coherence matrix.

2. The method of claim 1, wherein the first set of biomarkers are rs-fMRI (resting-state functional magnetic resonance imaging) waveforms obtained by measuring on each of the thalamic sub-nuclei of the patient.

3. The method of claim 1, wherein the postconsussive symptom score is a score of Rivermead postconcussive symptom questionnaire.

4. The method of claim 2, further comprising:
calculating coherence between every two of the thalamic sub-nuclei; and
arranging the calculated coherences into a first two-dimensional array to obtain the first coherence matrix.

5. The method of claim 4, further comprising:
receiving a second set of biomarkers of a plurality of cortical regions of the patient;
calculating a second coherence matrix from the second set of biomarkers; and
predicting the postconcussive symptom score of the patient for the given time through the machine learning-based predictive model based on the first coherence matrix and the second coherence matrix.

6. The method of claim 5, wherein the second set of biomarkers are rs-fMRI (resting-state functional magnetic resonance imaging) waveforms obtained by measuring on each of the cortical regions of the patient.

7. The method of claim 6, further comprising:
calculating coherence between every two of the cortical regions; and
arranging the calculated coherences into a second two-dimensional array to obtain the second coherence matrix.

8. The method of claim 5, further comprising:
receiving information about age and sex of the patient; and
predicting the postconcussive symptom score of the patient for the given time through the machine learning-based predictive model based on the first coherence matrix, the second coherence matrix, and the received information.

9. The method of claim 8, wherein a first average value of first elements in the first coherence matrix and a second average of second elements in the second coherence matrix are input to the machine learning-based predictive model to predict the postconcussive symptom score of the patient.

10. The method of claim 8, wherein the first coherence matrix and the second coherence matrix are input to the machine learning-based predictive model to predict the postconcussive symptom score of the patient.

11. An apparatus for predicting persistent post-concussive neuropsychiatric symptoms based on thalamocortical coherence, the apparatus comprising:
at least one memory having computer executable instructions stored therein; and
at least one processor coupled to the at least one memory,
wherein the computer executable instructions cause the at least one processor to perform operations, and the operations comprise:
receiving a first set of biomarkers of a plurality of thalamic sub-nuclei of a patient;
calculating a first coherence matrix from the first set of biomarkers; and
predicting a postconcussive symptom score of the patient for a given time through
a machine learning-based predictive model based on the first coherence matrix.

12. The apparatus of claim 11, wherein the first set of biomarkers are rs-fMRI (resting-state functional magnetic resonance imaging) waveforms obtained by measuring on each of the thalamic sub-nuclei of the patient.

13. The apparatus of claim 11, wherein the postconsussive symptom score is a score of Rivermead postconcussive symptom questionnaire.

14. The apparatus of claim 11, wherein the operations further comprise:
calculating coherence between every two of the thalamic sub-nuclei; and
arranging the calculated coherences into a first two-dimensional array to obtain the first coherence matrix.

15. The apparatus of claim 14, wherein the operations further comprise:
receiving a second set of biomarkers of a plurality of cortical regions of the patient;
calculating a second coherence matrix from the second set of biomarkers; and
predicting the postconcussive symptom score of the patient for the given time through the machine learning-based predictive model based on the first coherence matrix and the second coherence matrix.

16. The apparatus of claim 15, wherein the second set of biomarkers are rs-fMRI (resting-state functional magnetic resonance imaging) waveforms obtained by measuring on each of the cortical regions of the patient.

17. The apparatus of claim 16, wherein the operations further comprise:
calculating coherence between every two of the cortical regions; and
arranging the calculated coherences into a second two-dimensional array to obtain the second coherence matrix.

18. The apparatus of claim 15, wherein the operations further comprise:
receiving information about age and sex of the patient; and
predicting the postconcussive symptom score of the patient for the given time through the machine learning-based predictive model based on the first coherence matrix, the second coherence matrix, and the received information.

19. The apparatus of claim 18, wherein the machine learning-based predictive model is a support vector machine, and a first average value of first elements in the first coherence matrix and a second average of second elements in the second coherence matrix are input to the machine learning-based predictive model to predict the postconcussive symptom score of the patient.

20. The apparatus of claim 18, wherein the machine learning-based predictive model is a convolutional neural network, and the first coherence matrix and the second coherence matrix are input to the machine learning-based predictive model to predict the postconcussive symptom score of the patient.
